(19) United States
(12) Patent Application Publication
(10) Pub. No.: US 2025/0205392 A1
(43) Pub. Date: Jun. 26, 2025

(54) DEVICE AND METHOD FOR ACCELERATING AND GUIDING VASCULARIZATION

(71) Applicant: The Penn State Research Foundation, University Park, PA (US)

(72) Inventors: Amir Sheikhi, University Park, PA (US); Mohammad Zaman Ataie, University Park, PA (US); Dino J. Ravnic, University Park, PA (US); Summer Horchler, University Park, PA (US)

(21) Appl. No.: 18/848,297

(22) PCT Filed: Mar. 29, 2023
(86) PCT No.: PCT/US2023/016659

§ 371 Date: Sep. 18, 2024

Related U.S. Application Data
(60) Provisional application No. 63/324,774, filed on Mar. 29, 2022.
(60) Provisional application No. 63/367,521, filed on Jul. 1, 2022.
(60) Provisional application No. 63/424,286, filed on Nov. 10, 2022.

(51) Int. Cl. A61L 27/36; A61L 27/18; B33Y 80/00; C07C 57/04

(52) U.S. Cl. CPC A61L 27/3695; A61L 27/18; B33Y 80/00; C07C 57/04; A61L 2400/06; A61L 2430/34

(57) ABSTRACT

Embodiments relate to a device and/or method for vascularization, which can also be used to enhance regenerating tissues. The method comprises inducing surgical micropuncture, in which small perforations are created using a microscale needle in a wall of a target vessel. The method further comprises guiding microvascular development using granular and/or porous scaffolds, which are formed by converting materials, including polymers, to microparticles or porous constructs and subsequently assembling the microparticles or directly using the porous constructs by implanting on the target vessel and/or in the target tissue. The granular and/or porous scaffolds can also be formed on target tissues in situ.

### CROSS-REFERENCE TO RELATED APPLICATIONS

This patent application is related to and claims the benefit of priority of U.S. provisional application 63/424,286, filed on Nov. 10, 2022, the entire contents of which is incorporated by reference, and is further related to and claims the benefit of priority of U.S. provisional application 63/367,521, filed on Jul. 1, 2022, the entire contents of which is incorporated by reference, and is further related to and claims the benefit of priority of U.S. provisional application 63/324,774, filed on Mar. 29, 2022, the entire contents of which is incorporated by reference.

### STATEMENT REGARDING FEDERALLY SPONSORED RESEARCH DEVELOPMENT

This invention was made with government support under Grant No. EB032672 awarded by the National Institutes of Health. The Government has certain rights in the invention.

### FIELD OF THE INVENTION

Embodiments relate to revascularization using hydrogel-based scaffolds, and particularly to pattern vascularization using granular scaffolds and to accelerate vascularization via surgical micropuncture.

### BACKGROUND OF THE INVENTION

Tissue deficiencies can occur after a traumatic event or oncologic resection. Patients often suffer from infection, immobility, and dysfunction, and severe injuries, loss of limb and even life may occur. Over the past two decades, hydrogel-based scaffolds have become vital to tissue reconstruction by providing a base for revascularization. Thus, surgeons have widely incorporated hydrogel-based technologies into patient care. Unfortunately, hydrogel scaffolds are still plagued by some major limitations, including slow and random vascularization. Slow or inadequate vascular integration can lead to suboptimal outcomes, including seroma, infection, and reconstructive failure. Surgeons have attempted to prime the wound bed for angiogenesis using a variety of approaches, such as arteriovenous loops (AVLs). However, the induced vasculature still takes several weeks to form. This is suboptimal for wound reconstruction and prohibits true tissue regeneration.

A novel microsurgical approach termed micropuncture (MP) has recently been developed that significantly expedites scaffold vascularization. However, the MP-induced neovasculature in bulk hydrogels is random in nature, which does not fulfill the requirements of reconstructive surgeons where form dictates function. It is well-known that the structural characteristics of scaffolds profoundly affect cell infiltration and vascular ingrowth. It has been recognized that bulk hydrogels do not allow immediate cell infiltration, hence vascularization, because they lack interconnected pores, have nanoscale pores that are about three orders of magnitude smaller than cell size, and require degradation and remodeling that may take weeks, if not months. These shortcomings impede bulk hydrogel scaffolds from achieving optimally swift and patterned vascularization.

Architecturally, microengineered void spaces in hydrogel scaffolds is a cue for neovascularization. Interconnected pores enhance cell migration and proliferation, perfusability, and nutrient/oxygen transfer, which are all pivotal factors in vascularization and tissue growth. By tuning pore characteristics, such as size, interconnectivity, distribution, shape/architecture, and surface topography, properties of vascular networks are regulated. Different types of biomaterials, such as polyethylene glycol (PEG), gelatin, alginate, and tricalcium phosphate have been used for making porous scaffolds using particle leaching, 3D (bio) printing, templating, or freeze-drying. These methods are based on post-processing and often yield random pore networks or cause non-interconnected void spaces, which may limit physiologic vascularization. There has been virtually no success in creating a living vasculature in the microscale range. Simply put, this is not how the body intends for angiogenesis to occur.

Ultimately, inducing rapidly patterned vascularization within biomaterials has profound implications for current clinical treatment paradigms and scaleup of regenerative engineering platforms. To address this long-standing challenge, the MP approach can be combined with granular scaffold GHS technology to hasten and pattern microvascular network formation.

### BRIEF SUMMARY OF THE INVENTION

Embodiments relate to a combination of recent advances in reconstructive microsurgery and hydrogel microfabrication technology to provide the first synergistic platform that enables rapid perfusability and microvasculature patterning within an implanted scaffold. Surgical micropuncture (MP) is induced such that small perforations are created using a microscale needle in the recipient vasculature to facilitate cellular extravasation and angiogenesis without causing thrombosis or significant hemorrhage, followed by guiding microvascular development using adjacent granular scaffolds with well-defined microporosity formed via the photoassembly of microparticles. It is speculated that this technology opens unprecedented opportunities to redefine the tissue vascularization landscape with widespread applicability across all anatomic sites and disease etiology, including many cardiovascular-related pathologies, the leading cause of morbidity and mortality worldwide.

In an exemplary embodiment, a method and/or device for regenerating tissue and/or inducing vascularization comprises perforating a wall of a target vessel; and/or implanting a granular scaffold on a target tissue and/or the target vessel.

In some embodiments, the method further comprises, prior to implanting the granular scaffold on the target tissue and/or the target vessel, converting polymers to form microparticles via physical and/or chemical crosslinking; and assembling the microparticles to form the granular scaffold via physical and/or chemical bond formation.

In some embodiments, a shape of the microparticles is selected from the group consisting of spheres, cubes, cuboids, cones, cylinders, pyramids, and/or prisms.

In some embodiments, the microparticles are selected from the group consisting of hydrogels, plastics, elastomers, thermosets, and other polymers.

In some embodiments, the method further comprises injecting the microparticles at the target tissue and/or the target vessel prior to assembling the microparticles.

In some embodiments, the polymers are selected from the group consisting of proteins, peptides, carbohydrates, and lipids.

In some embodiments, the polymer is gelatin methacryloyl.

In some embodiments, the granular scaffold has a mean pore diameter in the order of a cell size of the target tissue and/or the target vessel.

In some embodiments, the granular scaffold has a mean pore diameter of 0.1-1000 μm.

In some embodiments, the granular scaffold has a void fraction between 20-25%. In some embodiments, the microparticles adhere to 1-10,000 cells.

In some embodiments, the granular scaffold has a tissue adhesion strength of 1-500 kPa.

In some embodiments, the method is configured to facilitate vascularization of the granular scaffold within 1-60 days, preferably 7-28 days, of implanting the granular scaffold on the target tissue and/or the target vessel.

In some embodiments, the step of perforating the wall of the target vessel comprises perforating the wall with a needle with a diameter of 1-2000 μm.

In some embodiments, the granular scaffold is porous with or without the use of microparticles.

In some embodiments, the granular scaffold is prefabricated or in situ fabricated on the target issue.

In some embodiments, the granular scaffold is additively manufactured via 3D printing.

In some embodiments, the granular scaffold is used for the regeneration, repair, reconstruction, and/or closure of soft and/or hard tissues with or without the step of perforating the wall of the target vessel.

In an exemplary embodiment, a method and/or device for inducing and/or patterning hierarchical vascularization comprises implanting a granular scaffold on a target tissue and/or a target vessel; and optionally perforating a wall of the target vessel.

Further features, aspects, objects, advantages, and possible applications of the present invention will become apparent from a study of the exemplary embodiments and examples described below, in combination with the Figures, and the appended claims.

### BRIEF DESCRIPTION OF THE FIGURES

The above and other objects, aspects, features, advantages, and possible applications of the present invention will be more apparent from the following more particular description thereof, presented in conjunction with the following drawings. It should be understood that like reference numbers used in the drawings may identify like components.

FIG. 1 shows an exemplary method of for regenerating tissue and/or inducing vascularization via a combination of granular hydrogel scaffolds and micropuncture.

FIG. 2A shows GelMA droplets converted to microgels via physical crosslinking at 4° C., followed by packing and chemical assembly via photocrosslinking to yield granular hydrogel scaffolds.

FIG. 2B shows optical microscopy images of small, medium, and large GelMA droplets.

FIG. 2C shows a droplet size frequency histogram based on n>2000 droplets, indicating highly monodispersed droplet formation with an average diameter of ≈29 μm (small), ≈81 μm (medium), or ≈173 μm (large).

FIG. 2D shows micropuncture performed on arteries and veins using a 60 μm microneedle at ≈1 mm intervals, followed by the implantation of granular hydrogel scaffolds adjacent to micropuncture vessels to guide vascular network architecture.

FIG. 2E shows small, medium, or large microgel building blocks yield granular hydrogel scaffolds with varying pore microarchitectures that guide the scattering, diameter, and length of blood vessels.

FIG. 3A shows assembly of small, medium, and large microgels yielding granular hydrogel scaffolds with tailored pore features, imaged at varying heights (Z-stacks) and in 3D.

FIG. 3B shows the effect of microgel size on granular hydrogel scaffold void fraction, defined as the volume ratio of interstitial void spaces to the total scaffold volume, showing that the void fraction is independent of the spherical building blocks.

FIG. 3C shows equivalent median pore diameter, as an indicator of granular hydrogel scaffold pore size, increases by increasing microgel size.

FIG. 3D shows compressive stress-strain curves for granular hydrogel scaffolds comprising small, medium, or large microgels alongside the bulk hydrogel counterpart (GelMA concentration=10 wt. %) as a control.

FIG. 3E shows compressive modulus measured based on the slope of strain-stress curves in the elastic region (≈0.05-0.15 mm mm⁻¹).

FIG. 3F shows frequency sweep tests, performed on samples at 10⁻¹-10² rad s⁻¹ and a constant strain of 0.1%, i.e., the LVE region.

FIG. 3G shows the storage modulus of granular hydrogel scaffold and bulk hydrogel at 1 rad s⁻¹ and 0.1% of strain.

FIG. 4A shows oscillatory amplitude sweep test conducted at a constant frequency of 1 rad s⁻¹ to assess the LVR of GelMA for GHS-S.

FIG. 4B shows oscillatory amplitude sweep test conducted at a constant frequency of 1 rad s⁻¹ to assess the LVR of GelMA for GHS-M.

FIG. 4C shows oscillatory amplitude sweep test conducted at a constant frequency of 1 rad s⁻¹ to assess the LVR of GelMA for GHS-L.

FIG. 4D shows oscillatory amplitude sweep test conducted at a constant frequency of 1 rad s⁻¹ to assess the LVR of GelMA for bulk hydrogel scaffolds.

FIG. 5A shows a schematic of cells seeded in microporous granular hydrogel scaffolds assembled using varying GelMA microgel sizes compared with a bulk, nanoporous GelMA hydrogel scaffold.

FIG. 5B shows that GHS-S and more significantly GHS-M promote SVF network formation, whereas GHS-L does not yield any network, and the bulk GelMA hydrogel scaffold does not support SVF cell proliferation and 3D network formation.

FIG. 5C shows roundness of encapsulated SVF cells in granular hydrogel scaffolds or bulk scaffolds, which is ≈40-60% or 90%, respectively. SVF cells elongate more in GHS-M compared with GHS-S and GHS-L, and do not elongate in the bulk scaffold at all.

FIG. 5D shows the total projected area of SVF cells, which was significantly higher in GHS-M compared with GHS-L and bulk GelMA.

FIG. 5E shows the number of SVF cell network branches in the scaffolds, showing excellent branch formation in GHS-M, some branch formation in GHS-S, and no branches in GHS-L or bulk scaffold after 2 days of culture.

FIG. 6 shows low magnification fluorescence images of human SVF cells labels using the cell tracker, seeded, and cultured in granular hydrogel scaffolds. While GHS-S and GHS-M maintain almost all the cells, GHS-L does not hold the cells, and the majority of cells escape the scaffold because of the large void spaces (media equivalent pore diameter ≈45 μm). In GHS-L, the dashed lines indicate the scaffold border (scale bar is 500 μm).

FIG. 7A shows fluorescence microscopy images of GHS-M seeded with a low (2,000), medium (4,000), or high (8,000) number of cells per μL of microgel suspension and cultured for 2 days (scale bar is 200 μm).

FIG. 7B shows the calculated total projected area for low seeding density.

FIG. 7C shows the calculated total projected area for medium seeding density.

FIG. 7D shows the calculated total projected area for high seeding density.

FIG. 8 shows the effect of human SVF cell seeding density on network formation in 3D GelMA (10 wt. %) bulk hydrogels. The confined microenvironment did not allow cell proliferation and elongation (scale bar is 200 μm).

FIG. 9 shows a gross image of a 15 mm×10 mm×3 mm granular hydrogel scaffold (see dashed lines) placed on the femoral artery and vein. The top arrow shows the flow of distal femoral arterial blood flow, and the bottom arrow represents femoral venous return (scale bar is 1 cm).

FIG. 10A shows scaffolds stained against CD31 to demonstrate endothelial cells and a DAPI counterstain to delineate all nucleated cells. Granular hydrogel scaffolds without micropuncture (control) enabled some vascular formation. Synergizing granular hydrogel scaffolds with micropuncture resulted in significantly higher endothelial cell infiltration and vascularization across microgel sizes. The bulk GelMA hydrogel scaffold either without micropuncture (control) or with micropuncture underwent limited vascular formation.

FIG. 10B shows mean vascular density in the scaffolds, showing enhanced network formation in GHS-S and GHS-M with micropuncture. Micropuncture did not result in any significant difference in neovascularization using GHS-L or bulk hydrogel.

FIG. 10C shows the average vessel diameter of vascular networks formed in varying scaffolds, showing almost similar values for all the granular hydrogel scaffold groups.

FIG. 10D shows total tube length in varying scaffolds, showing that GHS-M with micropuncture promoted the formation of longest tubes. GHS-S and GHS-L yielded significantly lower tube length than micropuncture GHS-M.

FIG. 10E shows intercapillary distance in varying scaffolds, which are correlated directly with microgel size: the larger the microgels the higher the intercapillary distances in granular hydrogel scaffolds.

FIG. 10F shows the number of branches in varying scaffolds, showing that micropuncture GHS-M yields a significantly higher number of branches than any other study group

FIG. 11 shows an AI analysis of micropuncture granular hydrogel scaffold stained with CD31 (scale bar is 100 μm).

FIG. 12 shows an angiograph image of GHS-M on the micropuncture artery and vein at Day 7 (scale bar is 100 μm).

FIG. 13A shows that following scaffold explantation and staining with F4/80 and DAPI, micropuncture granular hydrogel scaffold had a significantly larger proportion of infiltrating macrophages across all microgel sizes compared with non-micropuncture granular hydrogel scaffold. Some macrophage infiltration was seen in granular hydrogel scaffold without micropuncture (control). Bulk GelMA either without micropuncture (control) or with micropuncture failed to enable significant macrophage accumulation

FIG. 13B shows the area of F4/80-stained cells, quantifying the extent of macrophage accumulation in the scaffolds. Micropuncture granular hydrogel scaffold explants underwent an increase in macrophage accumulation. Bulk hydrogels did not permit macrophage infiltration.

FIG. 13C shows a histological assessment of systemic toxicity in liver, kidney, and spleen after 7 days of GHS-M implantation. GHS-M had no adverse effects either clinically or histologically, following a one-week implantation period in rats.

FIGS. 14A-C show in situ fabrication of granular hydrogel scaffolds from thermoresistive GelMA microgels (scale bar is 200 μm).

FIGS. 15A-D show material and mechanical characterization of granular hydrogel scaffolds made from thermoresistive GelMA microgels.

FIGS. 16A-C show biocompatibility assessment of granular hydrogel scaffolds made from thermoresistive GelMA microgels.

### DETAILED DESCRIPTION OF THE INVENTION

The following description is of an embodiment presently contemplated for carrying out the present invention. This description is not to be taken in a limiting sense but is made merely for the purpose of describing the general principles and features of the present invention. The scope of the present invention should be determined with reference to the claims.

Embodiments relate to a combination of recent advances in reconstructive microsurgery and hydrogel microfabrication technology to provide the first synergistic platform that enables rapid perfusability and microvasculature patterning within an implanted scaffold. Surgical micropuncture (MP) is induced such that small perforations are created using a microscale needle in the recipient vasculature to facilitate cellular extravasation and angiogenesis without causing thrombosis or significant hemorrhage, followed by guiding microvascular development using adjacent granular scaffolds.

It is contemplated that perforations may be made with a needle with a diameter of 1-2000 μm, preferably 30-100 μm.

It is contemplated that the granular scaffold may prefabricated or in situ fabricated on a target tissue and/or a target vessel. It is further contemplated that the granular scaffold may be additively manufactured via 3D printing.

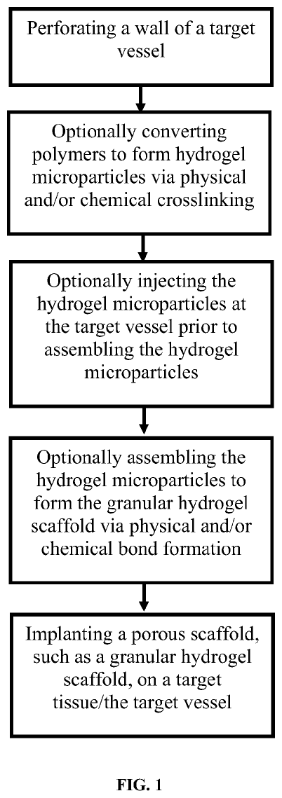

Referring to FIG. 1, embodiments relate to a method of regenerating tissue and/or inducing vascularization. The method comprises perforating a wall of a target vessel (e.g., MP) and subsequently implanting a granular scaffold on the target vessel and/or target tissue. The method may further comprise, prior to implanting the granular scaffold on the target vessel, converting polymers to form microparticles via physical and/or chemical crosslinking, and assembling the hydrogel microparticles to form the granular scaffold via physical and/or chemical bond formation (e.g., photoassembly). The method may further comprise injecting the microparticles at the target tissue/vessel prior to assembling the microparticles.

It is contemplated that vascularization of the granular scaffold may occur within 1-60 days, preferably 7-28 days, of implanting the granular scaffold on the target tissue and/or the target vessel.

It is contemplated that the granular scaffold may have favorable properties that promote vascular network formation with the target tissue, including but not limited to, microscale void spaces, patternable void spaces, tunable void fraction, cell-adhesive microgel building blocks, tissue adhesion, and pro-angiogenic cell signaling properties.

In particular, it is contemplated that the granular scaffold has a median pore diameter of 0.1-1000 μm, preferably 10-50 μm. It is further contemplated that the granular scaffold has a mean pore diameter in the order of a cell size of the target tissue and/or the target vessel. It is contemplated that the granular scaffold has patternable void spaces ranging from sub-micrometer to millimeter, preferably hierarchical pattern ranging from 10-50 μm. It is contemplated that the granular scaffold has a mean void fraction of 1-25%, preferably 20-25%. It is contemplated that the granular scaffold has cell-adhesive microgel building blocks that can adhere at 1-10,000 cells, preferably adhering 2-1000 cells. It is contemplated that the granular scaffold has a tissue adhesion strength of 1-500 kPa, preferably 10-100 kPa.

It is further contemplated that the granular scaffold has pro-angiogenic cell signaling properties, preferably signaling angiogenic pathways related to angiopoietin-TIE2 signaling and the VEGF family of ligands and receptor interactions.

It is contemplated that the granular scaffold may be configured to mimic the physiochemical and/or biological characteristics of the tissue. In particular, the granular scaffold may be configured to mimic the stiffness of the tissue.

In exemplary embodiments, the polymers may be any suitable polymers including protein-based materials and peptide-based materials. In particular, the polymers may be selected from the group consisting of any proteins, peptides, carbohydrates, lipids, or any other natural, semi-natural, and/or synthetic material. These can include hyaluronic acid, polyethylene glycol, and gelatin methacryloyl.

In exemplary embodiments, the polymers may be converted to form stable microparticles. The polymers may be converted to the microparticles via physical (thermal) crosslinking.

It is contemplated that the building blocks (e.g., microparticles) of granular scaffolds may be made of shapes selected from the list consisting of spheres, cubes, cuboids, cones, cylinders, pyramids, and prisms. In a preferred embodiment, the building blocks of granular scaffolds are spheres. It is further contemplated that the building blocks of granular scaffolds may be made using hydrogels, plastics, elastomers, thermosets, and other polymers. In a preferred embodiment, the building blocks of granular scaffolds are hydrogels.

It is contemplated that microparticles with any size and aspect ratio may be used. In a preferred embodiment, the size of the microparticles is between 10-200 μm. In a preferred embodiment, the aspect ratio of the microparticles is between 1-10.

It is contemplated that the terms “microgel” and “hydrogel microparticle” are used interchangeable throughout the present disclosure.

In exemplary embodiments, the microparticles are injected to an injection site within tissue. It is contemplated that the microparticles assemble via physical and/or chemical bond formation (e.g., photoassemble) to form the granular scaffold after injection into the tissue.

It is further contemplated that the microparticles may assemble to form granular scaffold after mixing with another polymer and/or colloidal particles. It is contemplated that this polymer may be aldehyde-modified carbohydrates and/or proteoglycans, including hyaluronic acid, protein and/or polymers in the extracellular matrix of native tissues, or another other suitable polymer that may form a hybridized granular scaffold.

It is further contemplated that the microparticles may assemble to form granular scaffolds after being decorated with biologics and/or colloidal particles and/or hybrid biologics-colloids. The surface of the microparticles may be coated with any biologics and/or the biologics may be encapsulated in the microparticles. The biologics may be biomolecules, growth factors (e.g., of hematopoietic growth factors, EGF, FGF, NGF, PDGF, VEGF, IGF, GMCSF, GCSF, TGF, Erythropieitn, TPO, BMP, HGF, GDF, Neurotrophins, MSF, SGF, and GDF and any other growth factors or biomacromolecules), cytokines, enzymatically modified DNA, drugs, peptides, or any combination thereof, or any other suitable biologics that may form granular scaffolds with enhanced bioactivity (e.g., a bioactive GHS). In exemplary embodiments, the biologics may be loaded (i.e., conjugated) to, attached on the surface of, or hybridized with nanocarriers bearing crosslinkable functional groups (e.g., vinyl groups). In exemplary embodiments, the biologics (e.g., the growth factors) are physically and/or chemically attached to colloids (e.g., heparin nanoparticles).

It is contemplated that cells fill the void spaces in the granular scaffolds and follow a similar pattern and/or hierarchical organization. Granular scaffold building blocks can be hierarchically patterned, for example using small (1-10 μm, preferably 5-10 μm), medium (10-30 μm, preferably, 20-25 μm), and large (30-100 μm, preferably 30-50 μm) microgels to induce patterned and/or hierarchical vascularization.

### Examples

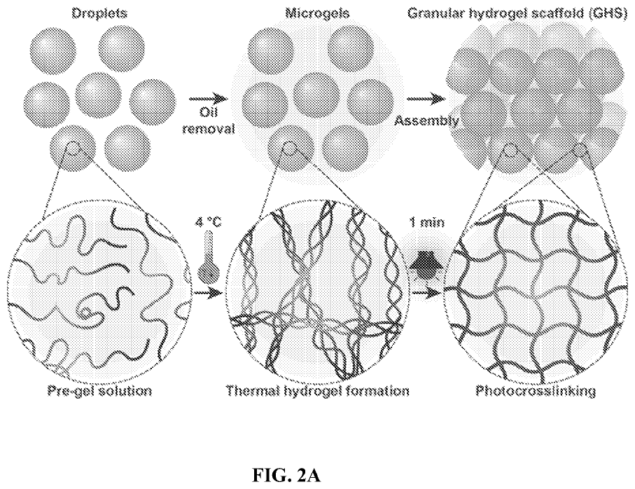

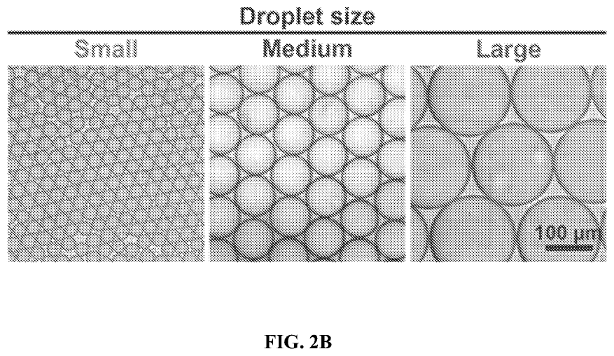

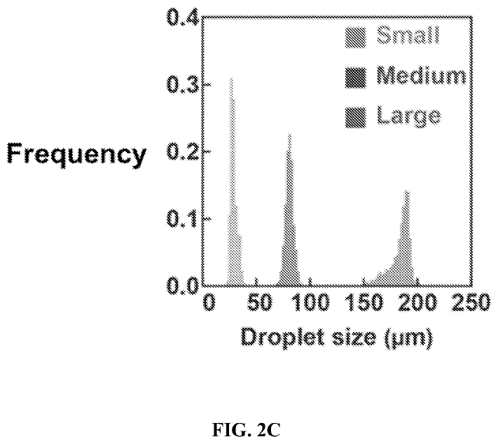

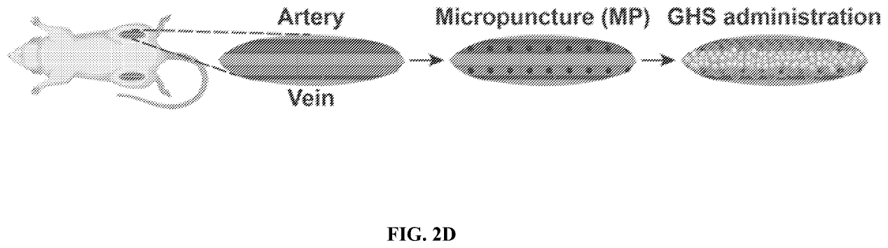

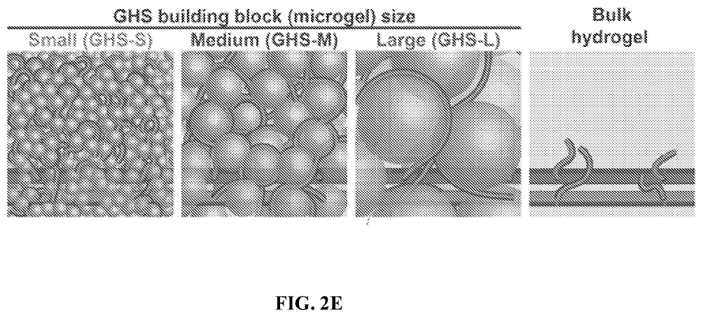

To form GHS building blocks, monodispersed droplets of a gelatin methacryloyl (GelMA) solution (10 wt. %) were fabricated as a dispersed phase in a continuous oil phase using a high-throughput step-emulsification microfluidic device, followed by microgels formation via physical (thermal) crosslinking at 4° C., as shown in FIG. 2A. The physical crosslinking stabilizes the microgels, enabling oil and surfactant removal and microgel transfer to aqueous media. Stabilized microgels were packed in an aqueous medium via centrifugation, and intra-particle crosslinking and inter-particle covalent bonding were established via the photo-induced free radical polymerization of GelMA vinyl groups, yielding the GHS. GelMA is a widely used biomaterial for engineering vascular networks as it bears a plenitude of cell-adhesive moieties, such as the arginylglycylaspartic acid (RGD) peptide motif, providing design flexibility to support endothelial cell adhesion and vessel formation. Gelatin-based hydrogels have commonly been used as protein-based biomaterials for microvasculature formation alongside collagen and fibrin, which permit network formation in micropatterned architectures or bioprinted constructs.

In the step-emulsification microfluidic devices, droplet size is regulated by the channel height. Devices with a step size of ≈8, ≈27, or ≈60 μm were fabricated to generate a range of droplet sizes, as shown in FIG. 2B.

FIG. 2C presents the size frequency histogram of small, medium, or large droplets, attaining an average diameter of 29±3 (small), 81±4 (medium), or 173±11 μm (large), respectively. These microgel building block sizes are designed to impart the GHS with median pore diameters that are in the order of mammalian cell size, such as the stromal vascular fraction (SVF) of adipose tissue, which includes endothelial progenitors and immune cells that play a key role in vascular network formation.

GHS may readily be used in combination with an established microsurgical approach, i.e., MP, in which blood vessels are punctured with an ultrafine microneedle to create precise perforations in the wall of a target recipient vessel. MP promotes rapid cell extravasation and accelerates adjacent microvasculature network formation. In this procedure, schematically shown in FIG. 2D, the hindlimb femoral artery and vein undergo MP, followed by the implantation of GHS on the vessels.

FIG. 2E shows the schematic illustration of GHS with small (GHS-S), medium (GHS-M), and large (GHS-L) microgel building blocks, alongside a bulk hydrogel scaffold, placed on the vein and artery after MP. It is hypothesized that the vascular microarchitecture may be guided by GHS pore characteristics.

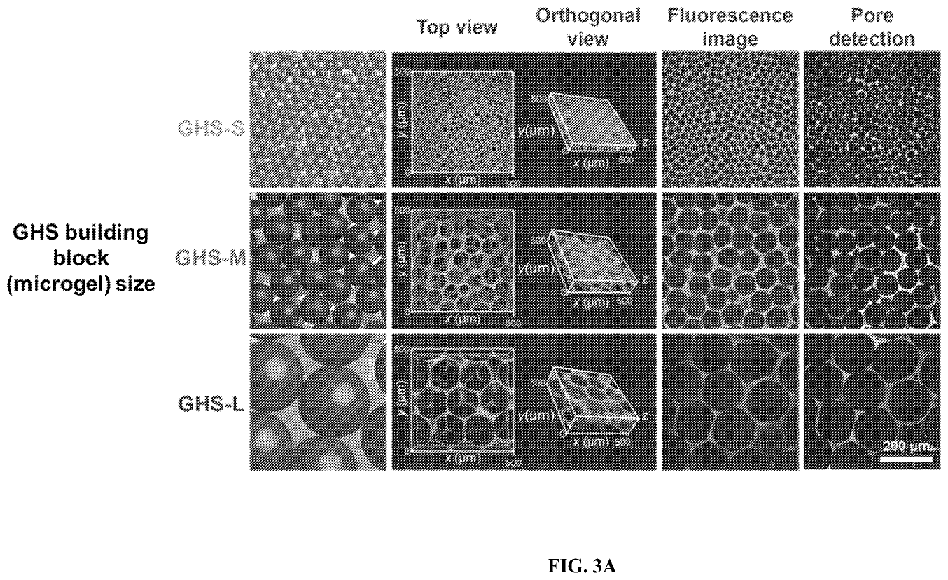

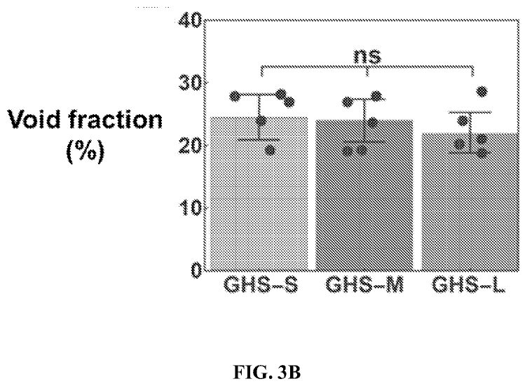

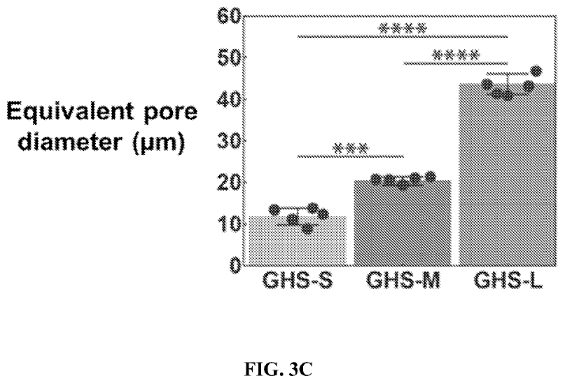

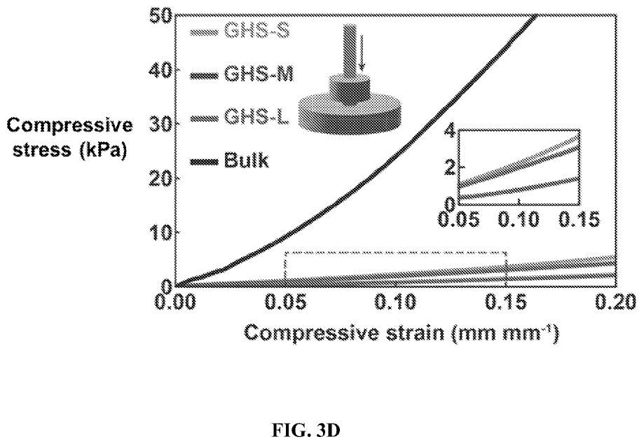

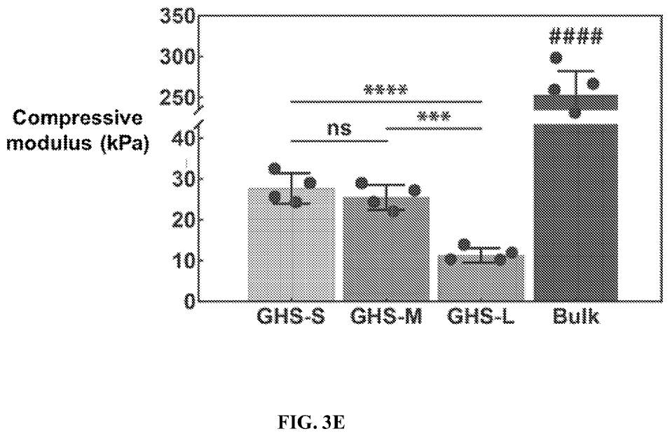

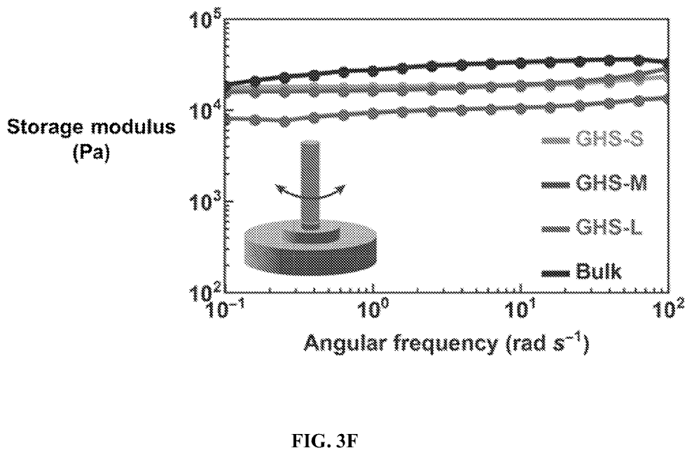

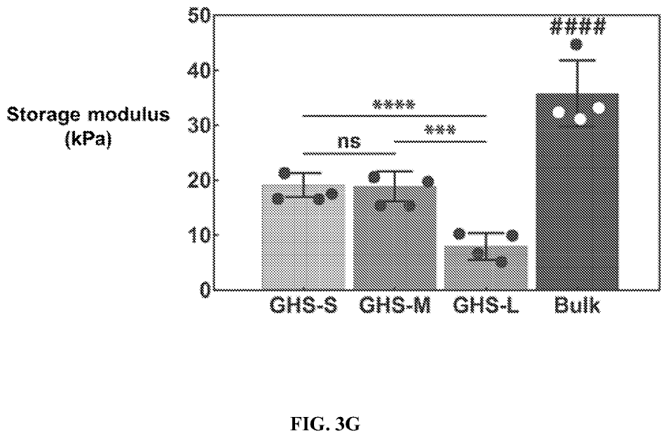

FIGS. 3A-3G present the pore features and mechanical properties of GHS, fabricated using varying sizes of microgels. High molecular weight fluorescent dextran molecules (≈2 MDa, equivalent Stokes diameter ≈54 nm) did not penetrate the microgels, labeling the interstitial void spaces of GHS. FIG. 3A shows the z-stacked and 3D fluorescence images of microporous GHS. Void fraction and equivalent median pore diameter were quantified via analyzing the 3D images. The volume ratio of void spaces to the total scaffold volume was reported as the void fraction in FIG. 3B. Void fraction of GHS≈20-25%, which was not significantly affected by varying spherical microgel sizes; however, the void spaces are enlarged by increasing the microgel size, as shown in FIG. 3C. Small, medium, or large microgels yielded GHS-S, GHS-M, or GHS-L with a median equivalent pore diameter of 12±2, 20±2, or 44±3 μm, respectively.

FIGS. 3D-3G show the mechanical characterization of GHS using compression and oscillatory rheology tests. FIG. 3D presents the compressive stress-strain curves of GHS and the bulk hydrogel counterpart comprising similar GelMA polymer concentration. At any compressive strain, the stress value of GHS was lower than the bulk hydrogel. This is a result of the highly porous GHS structure and limited contact area between adjacent microgels. The slope of stress-strain curves at the elastic region, typically at ≈0.05-0.15 mm mm⁻¹, was calculated and reported as the compressive modulus in FIG. 3E. The compressive modulus was significantly lower in GHS-L than GHS-S or GHS-M, possibly resulting from larger void space and weakened microgel-microgel contact among large microgels per unit volume. In addition, the compressive modulus of bulk hydrogel was about one order of magnitude higher than GHS, which also represents the local compressive modulus of microgels. The GHS stiffness was in the range of human soft tissues.

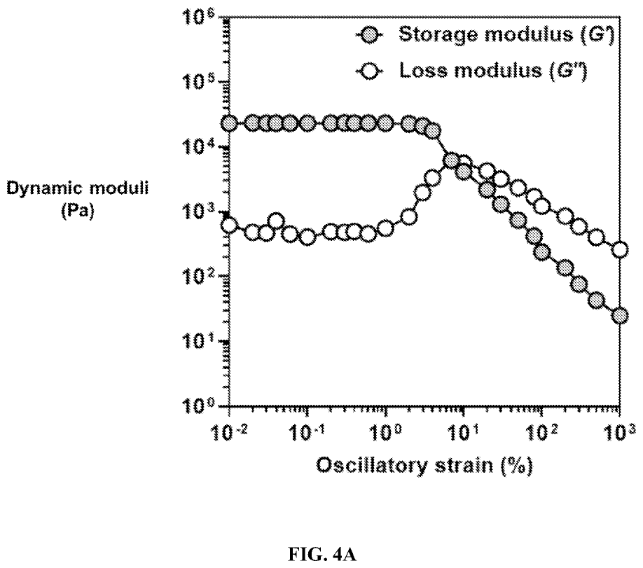

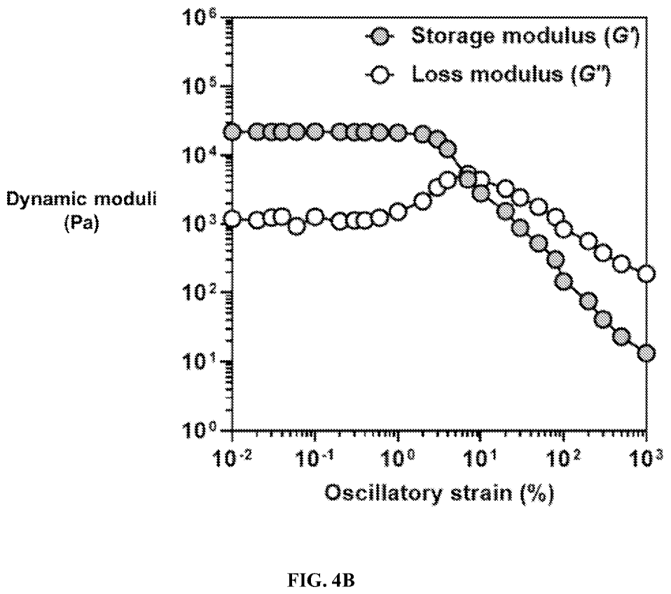

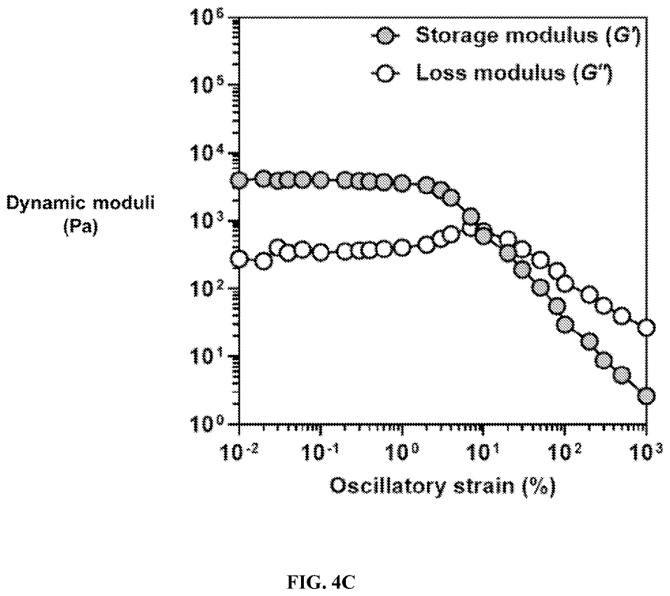

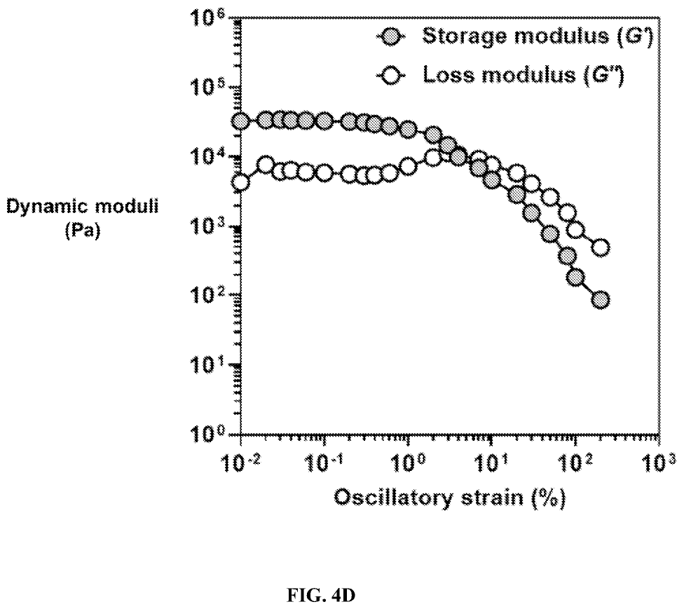

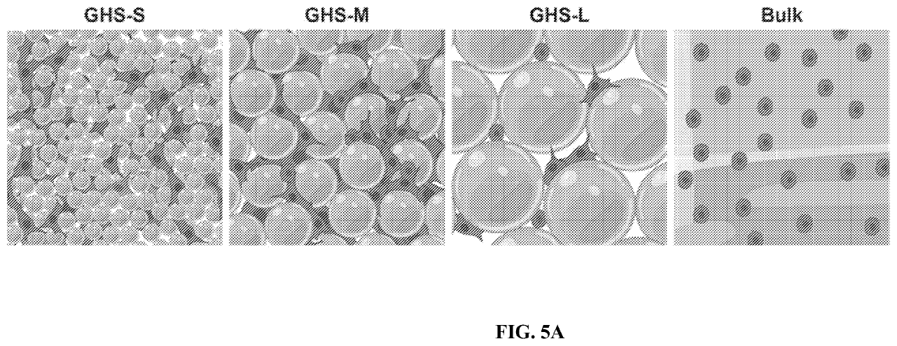

Viscoelastic properties of GHS were characterized via oscillatory rheology. Samples were assessed based on the oscillatory strain sweep ranging from 10⁻² to 10²% at a constant angular frequency of 1 rad s⁻¹ to determine the linear viscoelastic region (LVR) and flow point (see FIGS. 4A-4D). As the LVR for all the gels was below ˜ 1% of strain at 1 rad s⁻¹, the oscillatory frequency sweep was performed at 10⁻¹-10² rad s⁻¹ and a constant strain of 0.1% (see FIG. 3F). The storage modulus for GHS-L was lower than GHS-S or GHS-M, and the storage modulus of bulk hydrogel was higher than all GHS, as presented in FIG. 3G. Overall, the storage modulus had a similar trend as the compressive modulus.

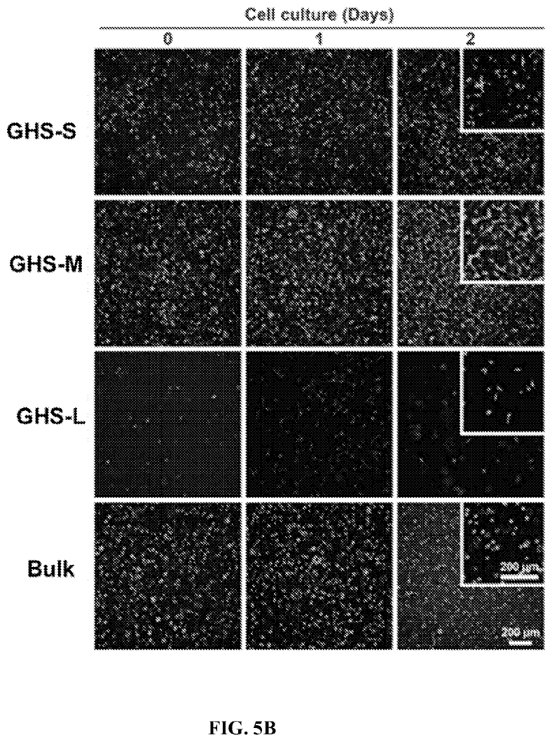

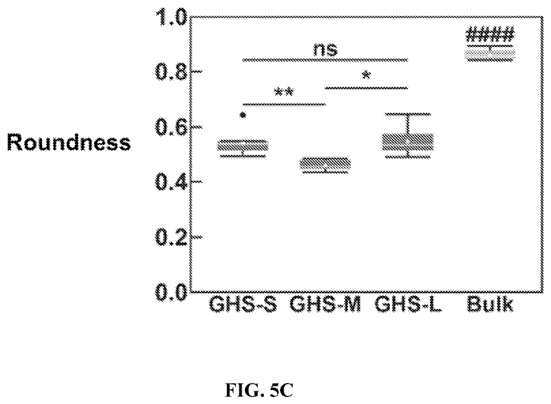

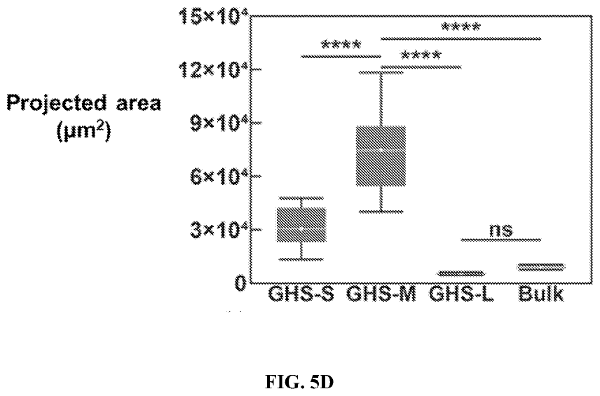

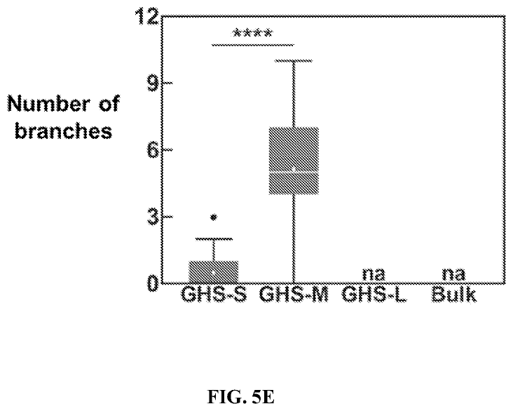

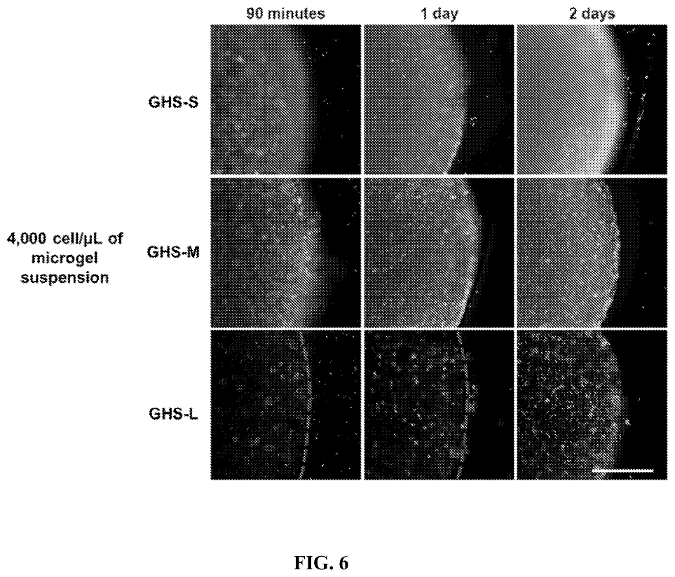

To examine the effect of scaffold microarchitecture on in vitro cell activity, GHS with tailored microscale pores were seeded with the human SVF cells and compared with the bulk hydrogel counterpart (nanoporous, GelMA concentration=10 wt. %), as schematically presented in FIG. 5A. The time-dependent behavior of SVF cells cultured in microvascular endothelial growth media is shown in FIG. 5B. While the cells remained non-prolific, round-shaped, and incapable of network formation in the bulk GelMA hydrogel, they were able to readily spread in GHS. Among GHS, GHS-L was not able to maintain all the SVF cells among the microgels because the pores were too large compared with the cell size. FIG. 6 shows that within 90 minutes of seeding, GHS-S and GHS-M hold almost all the cells, whereas the majority of cells are entrained out of GHS-L.

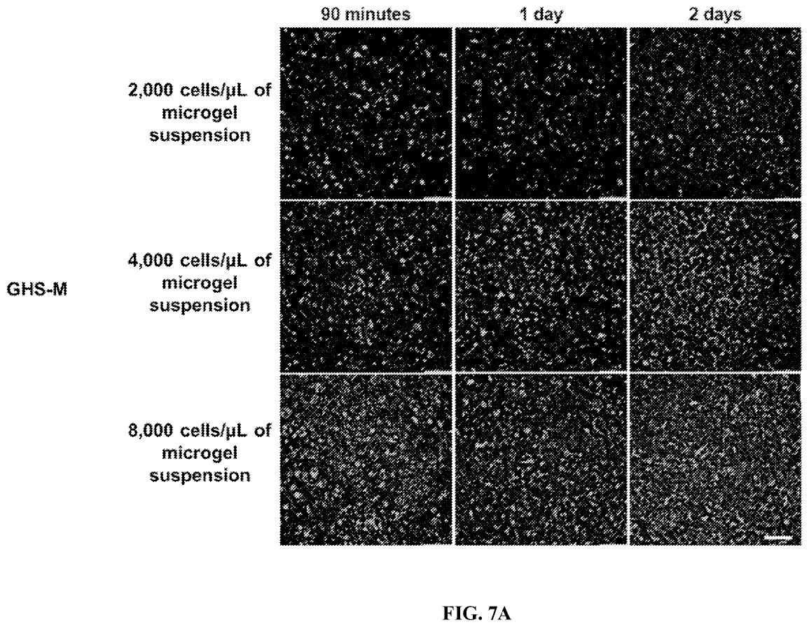

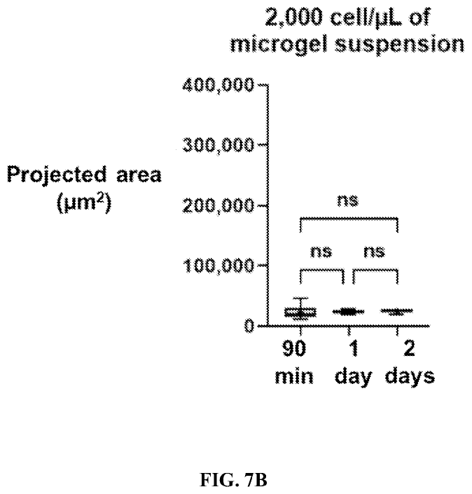

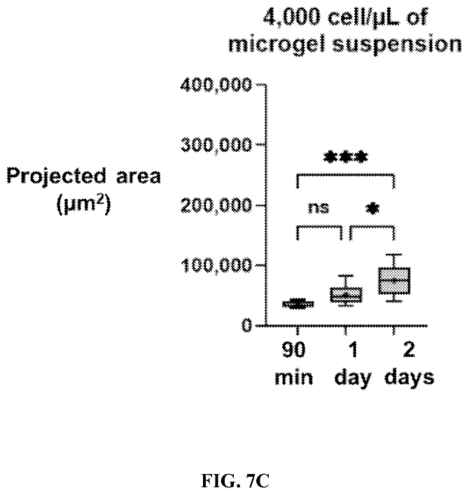

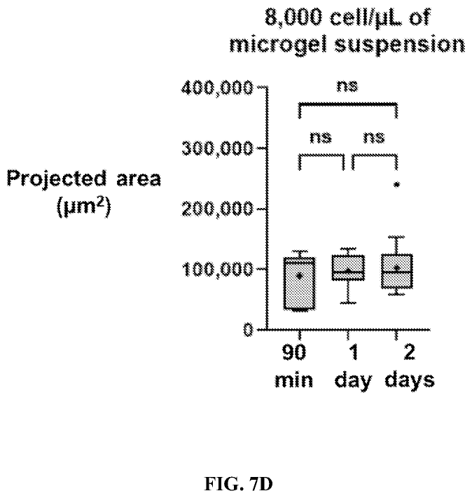

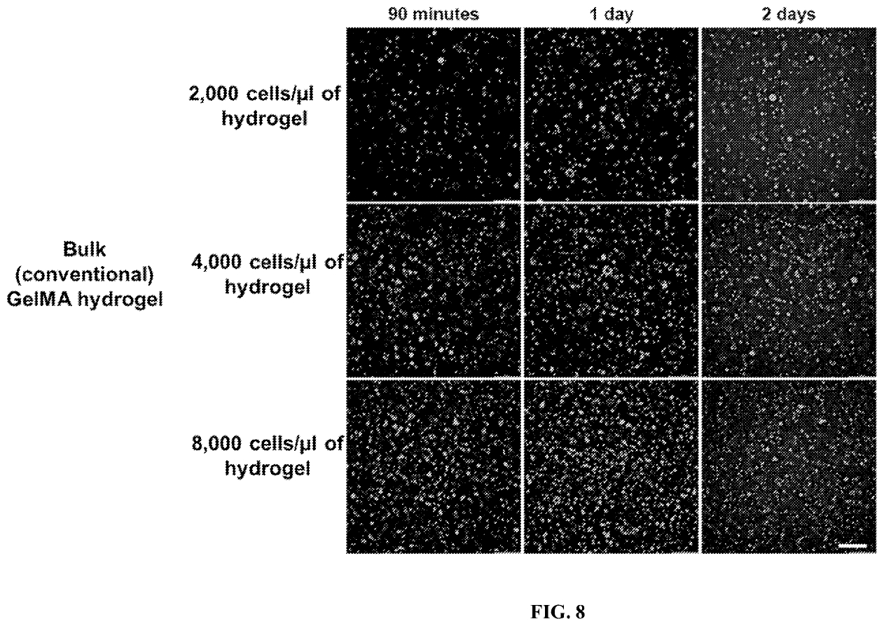

FIG. 5C presents the roundness of cells in the scaffolds, showing that encapsulated cells in the bulk hydrogel had significantly higher roundness (0.86±0.02) compared with the elongated cells in GHS (GHS-S≈0.54±0.04, GHS-M≈0.45±0.01, GHS-L≈0.54±0.05). Importantly, cells started forming networks in GHS-S and GHS-M within 2 days; however, no network was observed in GHS-L. FIGS. 5D and 5E present the projected cell area and the number of network branches, respectively. Unlike the bulk hydrogel, microporous structure of GHS-S and GHS-M facilitated network formation via increasing cell coverage (projected area) and number of branches. The ability to form such networks is essential for successful in vivo vascularization. When the median pore size was significantly larger than cell size, i.e., in GHS-L, cells were not able to form any network, and cell-cell connections were weakened. Initial cell seeding density affected the network formation quality in GHS, as presented in FIGS. 7A-7D, which is also reported for bulk hydrogels. At an exceeding low or high seeding density, the cells were not able to from any network in the GHS, because of lack of cell-cell connection or becoming overconfluent, respectively. FIG. 8 presents the effect of SVF cell seeding density on network formation capability in the bulk GelMA hydrogel. Independent of cell seeding density, the bulk GelMA hydrogel (10 wt. %) was unable to support network formation because of impaired cell spreading and proliferation. These data shed light on why currently used bulk scaffolds in clinical settings are unable to provide an optimal vascularization platform. Accordingly, there exists an optimal pore size range in GHS that promotes cell network formation and, consequently vascularization.

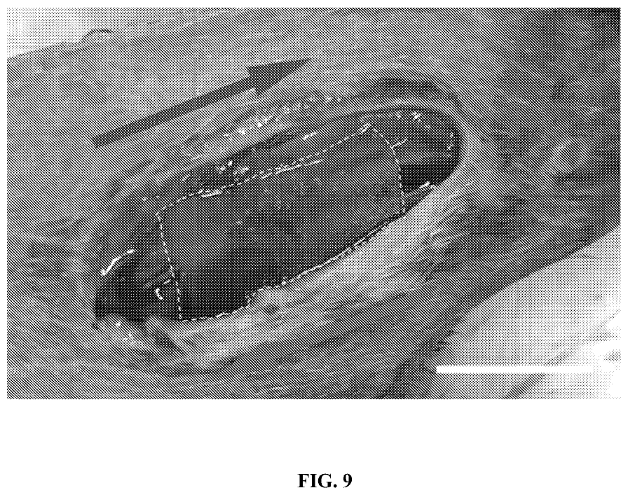

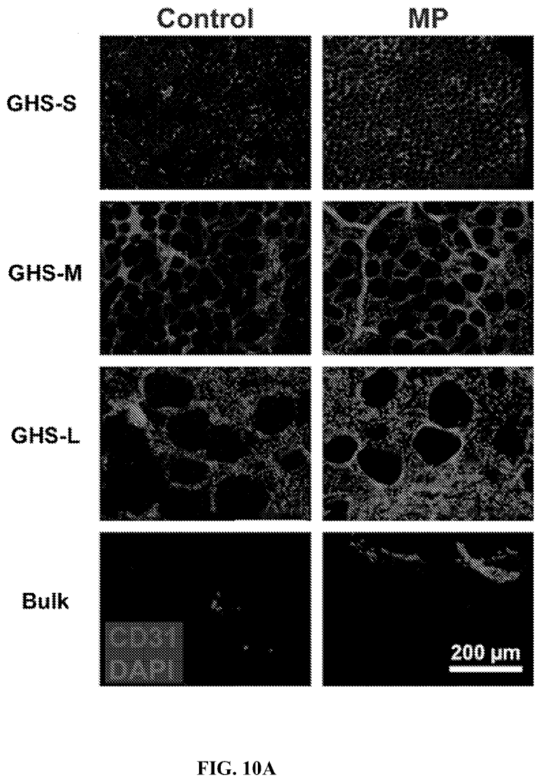

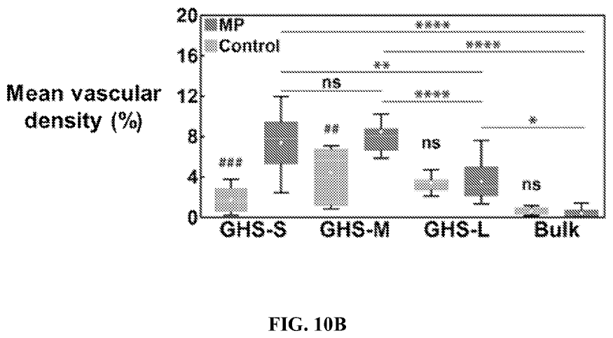

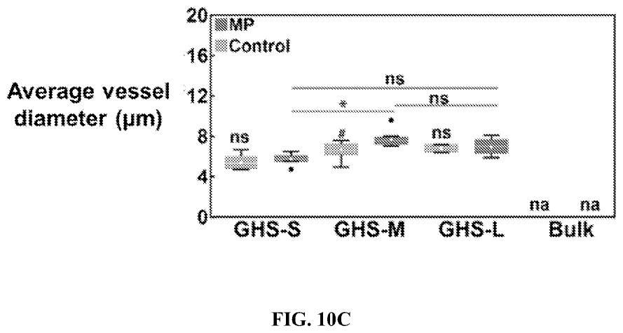

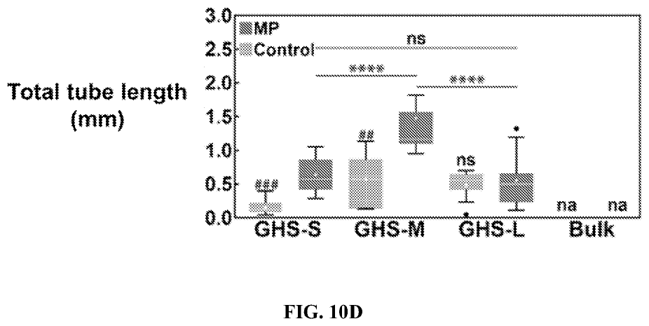

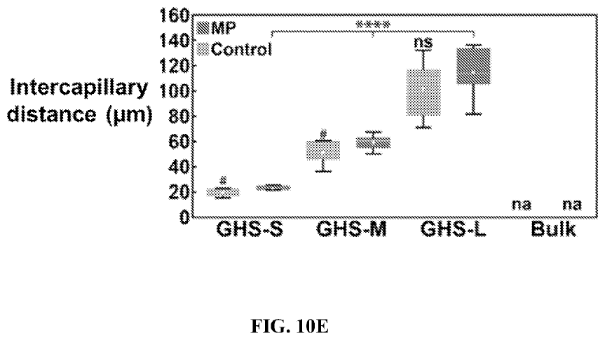

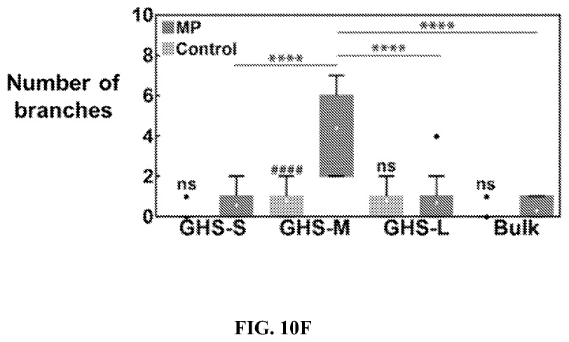

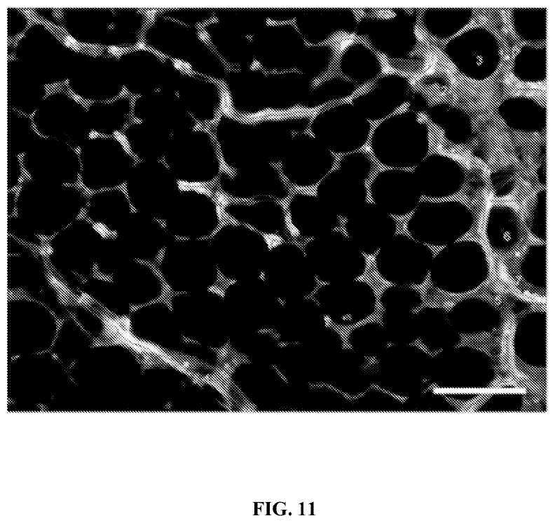

GHS or bulk hydrogel scaffolds were implanted adjacent to the MP artery and vein. FIG. 9 shows a gross image of an implanted scaffold after MP microsurgery. Implanted scaffolds in MP and non-MP (control) groups were analyzed at Day 7 to assess in vivo cellular infiltration and neovascular network formation, as presented in FIGS. 10A-10F. GHS placed adjacent to the MP artery and vein underwent an increase in cellular infiltration independent of microgel size. On the other hand, no significant cellular infiltration was observed in the implanted bulk hydrogels because of its nanoporous structure. MP allowed for rapid cellular extravasation into GHS, and GHS enabled accelerated inward cell infiltration. An increased accumulation of endothelial cells was observed lining MP scaffold pores, along with the formation of new microvascular capillary networks around the microgels (see FIG. 10A).

Accelerated neovascularization was patterned by using different sizes of microgel building blocks. Varying the GHS pore size caused differences in the accessible space for neovascular plexus formation originating from the adjacent femoral artery and vein. Using artificial intelligence (AI), the fluorescence images were analyzed and quantified (see FIG. 11).

FIG. 10B presents the mean vascular density of GHS and bulk scaffold implanted adjacent to MP or non-MP (control) vessels after 7 days. With MP, the vascular density of GHS-M and GHS-S was significantly higher than GHS-L and bulk. Results showed the significance of MP in neovascularization, especially in GHS-S and GHS-M. GHS-L did not show any significant difference with MP versus without MP, possibly due to the extra-large void spaces, which bypass the vessel formation capacity. Results show that the mean vascular density is significantly impacted by the synergistic interactions between pore features and MP (as seen in Table 1).

TABLE 1
Adjusted p-values of Tukey's multiple comparisons
test after two-way ANOVA for the mean vascular density

| Control | | | MP | | | | | |
|---|---|---|---|---|---|---|---|---|
| GHS-M | GHS-L | Bulk | GHS-S | GHS-M | GHS-L | Bulk | | |
| p = | p = | p = | ****p < | ****p < | p = | p = | GHS-S | Control |
| 0.0774 | 0.6408 | 0.9152 | 0.0001 | 0.0001 | 0.5276 | 0.8316 | | |
| | p = | **p = | *p = | **p = | p = | ***p = | GHS-M | |
| | 0.9353 | 0.0019 | 0.0488 | 0.0011 | 0.9716 | 0.0009 | | |
| | | p = | **p = | ****p < | p > | *p = | GHS-L | |
| | | 0.0656 | 0.013 | 0.0001 | 0.9999 | 0.0385 | | |
| | | | ****p < | ****p < | *p = | p > | Bulk | |
| | | | 0.0001 | 0.0001 | 0.0420 | 0.9999 | | |
| | | | | p = | **p = | ****p < | GHS-S | MP |
| | | | | 0.9197 | 0.0023 | 0.0001 | | |
| | | | | | ****p < | ****p < | GHS-M | |
| | | | | | 0.0001 | 0.0001 | | |
| | | | | | | *p = | GHS-L | |
| | | | | | | 0.0239 | | |

No significant vascular ingrowth was obtained in the adjacent bulk GelMA scaffold, and the results could not be analyzed due to vessel scarcity. Under these conditions it appeared that even though MP provided a rapid route of cellular extravasation from the targeted vasculature, the bulk hydrogel scaffold characteristics were insufficient to support cell infiltration. Increased endothelial cell infiltration (panel A) appeared concordant with the vascular density observed in MP GHS.

FIG. 10C presents average vessel diameter, which was in the range of 4-10 μm among the GHS groups. Statistical analyses show that the size of newly formed vessels is not MP dependent. However, void space size may slightly affect the vessel diameter (see Table 2).

TABLE 2
Adjusted p-values of Tukey's multiple comparisons test
after two-way ANOVA for the average vessel diameter

| Control | | MP | | | | |
|---|---|---|---|---|---|---|
| GHS-M | GHS-L | GHS-S | GHS-M | GHS-L | | |
| p = | p = | p = | **p = | p = | GHS-S | Control |
| 0.2448 | 0.1734 | 0.9987 | 0.0015 | 0.1043 | | |
| | p > | p = | p = | p > | GHS-M | |
| | 0.9999 | 0.4945 | 0.5929 | 0.9999 | | |
| | | p = | p = | p > | GHS-L | |
| | | 0.6094 | 0.7053 | 0.9999 | | |
| | | | *p = | p = | GHS-S | MP |
| | | | 0.0106 | 0.3525 | | |
| | | | | p = | GHS-M | |
| | | | | 0.8339 | | |

FIG. 10D shows that the tube length was significantly higher in MP GHS-M due to the significant expansion of small branching outgrowths and neovessel formation. MP and void space size significantly affected total scaffold tube length (see Table 3).

TABLE 3
Adjusted p-values of Tukey's multiple comparisons
test after two-way ANOVA for the total tube length

| Control | | MP | | | | |
|---|---|---|---|---|---|---|
| GHS-M | GHS-L | GHS-S | GHS-M | GHS-L | | |
| p = | p = | p = | ****p < | p = | GHS-S | Control |
| 0.1909 | 0.4568 | 0.0688 | 0.0001 | 0.2371 | | |
| | p = | p = | ****p < | p > | GHS-M | |
| | 0.9996 | 0.9998 | 0.0001 | 0.9999 | | |
| | | p = | ****p < | p > | GHS-L | |
| | | 0.9799 | 0.0001 | 0.9999 | | |
| | | | ****p < | p = | GHS-S | MP |
| | | | 0.0001 | 0.9993 | | |
| | | | | ****p < | GHS-M | |
| | | | | 0.0001 | | |

As presented in FIG. 10E and Table 4, intercapillary distance was a function of spacing dictated by GHS building block size, which was independent of MP. This shows that the vascular network can readily be patterned by GHS microarchitecture in vivo.

TABLE 4
Adjusted p-values of Tukey's multiple comparisons test
after two-way ANOVA for the intercapillary distance

| Control | | MP | | | | |
|---|---|---|---|---|---|---|
| GHS-M | GHS-L | GHS-S | GHS-M | GHS-L | | |
| ****p < | ****p < | p = | ****p < | ****p < | GHS-S | Control |
| 0.0001 | 0.0001 | 0.9892 | 0.0001 | 0.0001 | | |
| | ****p < | ****p < | p = | ****p < | GHS-M | |
| | 0.0001 | 0.0001 | 0.6054 | 0.0001 | | |
| | | ****p < | ****p < | p = | GHS-L | |
| | | 0.0001 | 0.0001 | 0.3636 | | |
| | | | ****p < | ****p < | GHS-S | MP |
| | | | 0.0001 | 0.0001 | | |
| | | | | ****p < | GHS-M | |
| | | | | 0.0001 | | |

The total number of branches in the field of view is shown in FIG. 10F, indicating significantly pronounced neovascular network formation using MP GHS-M. All other groups, including non-MP (control) groups (see Table 5), did not yield significant branch numbers, whether due to extremely small or large pores.

TABLE 5
Adjusted p-values of Tukey's multiple comparisons
test after two-way ANOVA for the number of branches

| Control | | | MP | | | | | |
|---|---|---|---|---|---|---|---|---|
| GHS-M | GHS-L | Bulk | GHS-S | GHS-M | GHS-L | Bulk | | |
| p = | p = | p > | p = | ****p < | p = | p > | GHS-S | Control |
| 0.9958 | 0.9695 | 0.9999 | 0.9958 | 0.0001 | 0.9873 | 0.9999 | | |
| | p > | p = | p > | ****p < | p > | p = | GHS-M | |
| | 0.9999 | 0.9873 | 0.9999 | 0.0001 | 0.9999 | 0.9958 | | |
| | | p = | p > | ****p < | p > | p = | GHS-L | |
| | | 0.9873 | 0.9999 | 0.0001 | 0.9999 | 0.9958 | | |
| | | | p = | ****p < | p = | p > | Bulk | |
| | | | 0.9990 | 0.0001 | 0.9958 | 0.9999 | | |
| | | | | ****p < | p > | p = | GHS-S | MP |
| | | | | 0.0001 | 0.9999 | 0.9998 | | |
| | | | | | ****p < | ****p < | GHS-M | |
| | | | | | 0.0001 | 0.0001 | | |
| | | | | | | p = | GHS-L | |
| | | | | | | 0.9990 | | |

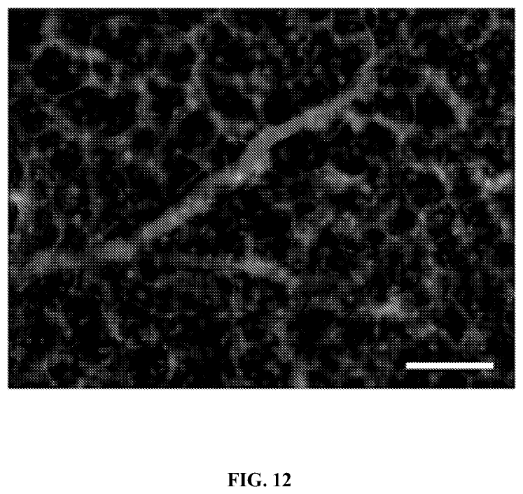

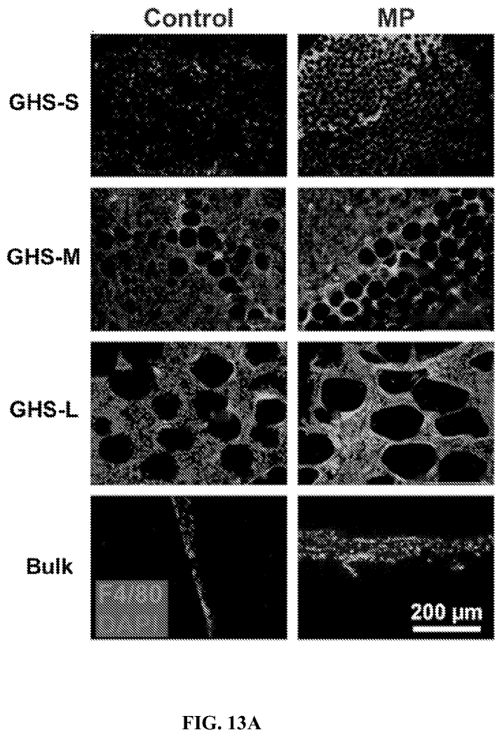

FIG. 12 shows an angiogram of vascularized GHS-M at Day 7, indicating perfusability of the formed vasculature. This confirmed vascular continuity between the recipient and scaffold microvasculature and further suggested that the formed vasculature was guided by the GHS pore microarchitecture.

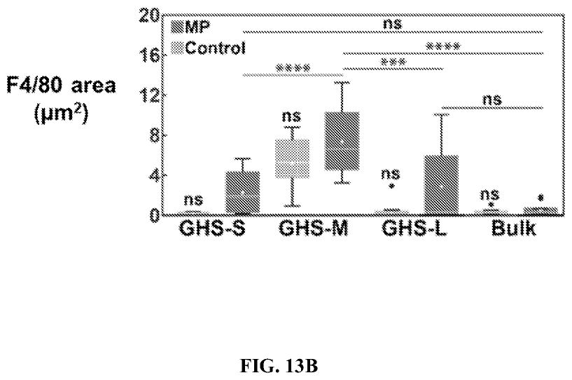

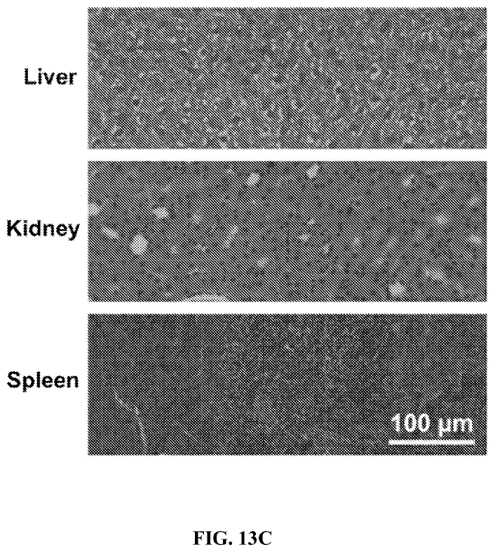

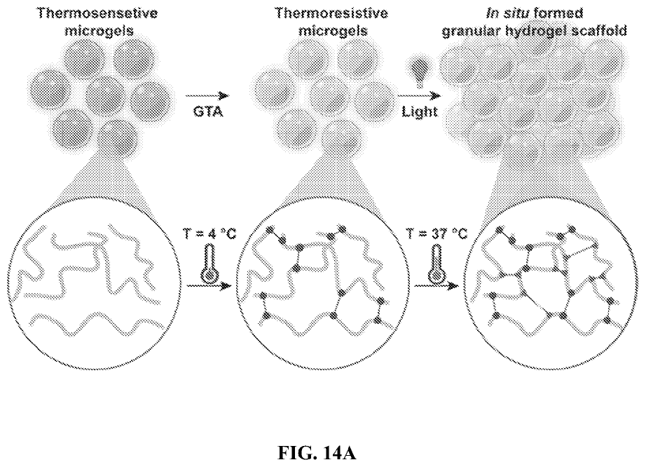

It has been demonstrated that macrophages are integral to angiogenesis. To understand their correlation to enhanced GHS vascularization after MP, scaffold macrophages were stained against F4/80 (green) and DAPI (blue) at Day 7, as shown in FIG. 13A. It was observed that vascularization in the GHS corresponded to early macrophage accumulation. Increased macrophage and total cellular infiltration were seen in all MP GHS when compared with the bulk hydrogel scaffold. FIG. 13B presents the area correlated to the stained macrophages across conditions (p-values may be found in Table 6).

TABLE 6
Adjusted p-values of Tukey's multiple comparisons
test after two-way ANOVA for the macrophage area

| Control | | | MP | | | | | |
|---|---|---|---|---|---|---|---|---|
| GHS-M | GHS-L | Bulk | GHS-S | GHS-M | GHS-L | Bulk | | |
| ***p = | p > | p > | p = | ****p < | p = | p > | GHS-S | Control |
| 0.0001 | 0.9999 | 0.9999 | 0.4568 | 0.0001 | 0.1436 | 0.9999 | | |
| | ***p = | ***p = | p = | p = | p = | ***p = | GHS-M | |
| | 0.0003 | 0.0002 | 0.0870 | 0.4031 | 0.3263 | 0.0005 | | |
| | | p > | p = | ****p < | p = | p > | GHS-L | |
| | | 0.9999 | 0.6035 | 0.0001 | 0.2274 | 0.9999 | | |
| | | | p = | ****p < | p = | p > | Bulk | |
| | | | 0.5655 | 0.0001 | 0.2032 | 0.9999 | | |
| | | | | ****p < | p = | p = | GHS-S | MP |
| | | | | 0.0001 | 0.9982 | 0.6915 | | |
| | | | | | p = | ****p < | GHS-M | |
| | | | | | 0.0009 | 0.0001 | | |
| | | | | | | p = | GHS-L | |
| | | | | | | 0.2922 | | |

The highest extent of scaffold macrophage infiltration was observed in GHS-M, which is consistent with the highest vascular density, total tube length, and the number of branches yielded in this scaffold. Accordingly, the optimum median pore size of GHS to maximize the quality of neovascularization is about 20 μm, a size that is comparable with the size of macrophages (10-20 μm) and endothelial cells (10-30 μm). Furthermore, the cytotoxicity of scaffolds was assessed clinically via weight, and there was no associated weight loss in any animal cohort. Histology was used to demonstrate end organ normalcy, as shown in FIG. 10C. No parenchymal injury was observed on liver, kidney, and spleen histology. No animals needed to be withdrawn from the study. Accordingly, GHS did not have any toxic side effects after the implantation, which is a key requirement for further translational considerations.

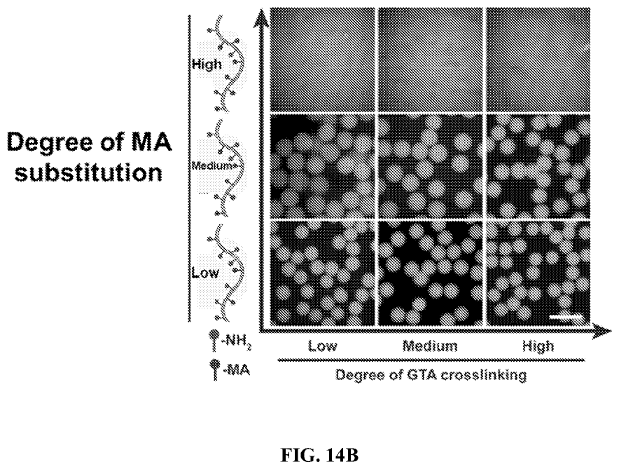

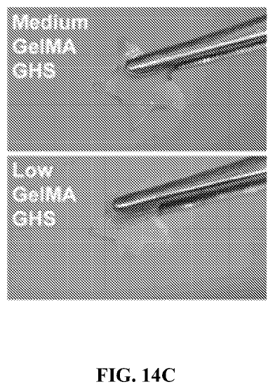

FIGS. 14A-C show in situ formation of GHS from thermoresistive GelMA microgels. FIG. 14A shows that GelMA hydrogel microparticles are thermoresponsive and unable to form a scaffold at body temperature (e.g., 37° C.). To overcome this limitation, the GelMA hydrogel microparticles was crosslinked via a Schiff-base reaction to produce thermoresistant microgels. These thermoresistive microgels served as building blocks for the in situ fabrication of GHS via radical photopolymerization.

FIG. 14B shows that GelMA was synthesized using three different degrees of substitutions (DOS) with high (˜70%), medium (˜40%) and low (˜10%), which affected the availability of primary amine groups for Schiff-base crosslinking using glutaraldehyde (GTA). High DoS resulted in insufficient primary amine groups, making the thermoresistive microgel unstable. In contrast, medium and low DoS resulted in stable thermoresistive microgels.

FIG. 14C shows thermoresistive microgels from medium and low GelMA were scaffolded to form a mechanically stable GHS.

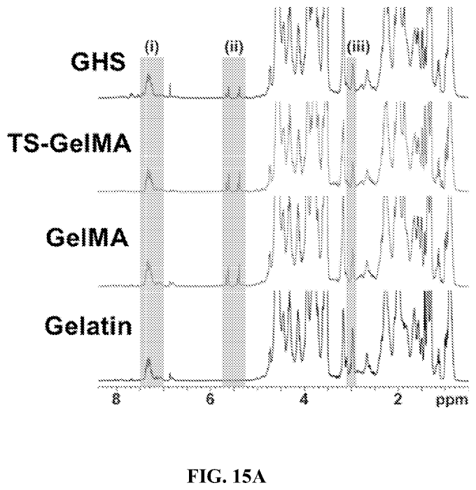

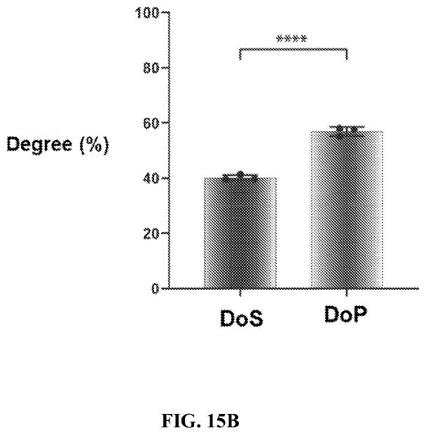

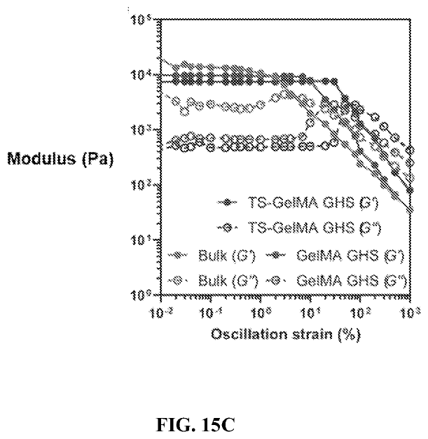

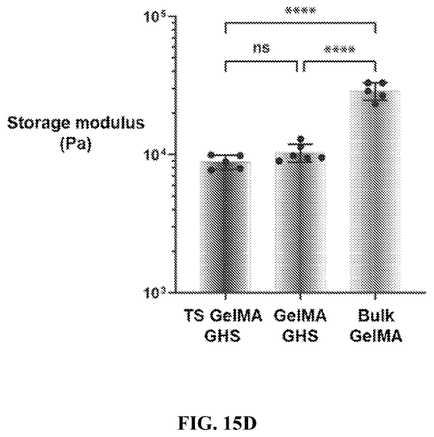

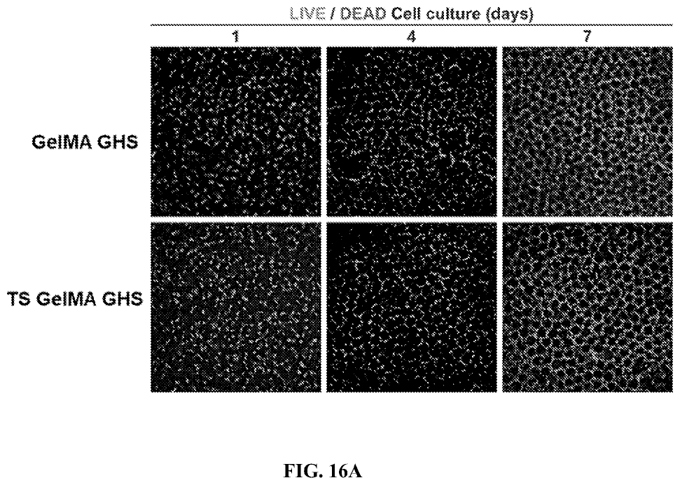

FIGS. 15A-D show material and mechanical characterization of GHS made from thermoresistant microgels. FIG. 15A shows ¹H NMR spectrometry of pure gelatin microgel, uncrosslinked GelMA microgel (with medium DoS), thermoresistant GelMA hydrogel microparticles, and photocrosslinked GHS composed of thermoresistant microgels. The peaks for aromatic acids (i) serve as the reference in all the groups. The presence of vinyl groups (ii) and a decrease in lysine protons (iii) were also observed.

FIG. 15B shows oscillatory strain sweep at constant frequency of 1 rad/s performed on in situ fabricated scaffolds: GHS from thermoresistant GelMA microgels (TS GelMA GHS), GHS from thermoresponsive GelMA microgels (GelMA GHS), and conventional (bulk) GelMA scaffolds.

FIG. 15C shows dynamic moduli versus oscillation strain for different scaffolds at a constant frequency of 1 rad/s, showing the storage and loss moduli of TS GelMA GHS, GelMA GHS, and conventional (bulk) GelMA scaffolds.

FIG. 15D shows the storage modulus measured at 1 rad/s and 0.1% strain. No significant difference was observed between the TS GelMA GHS and GelMA GHS, but bulk GelMA had significantly higher stiffness.

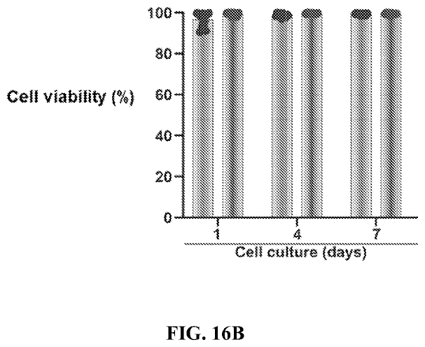

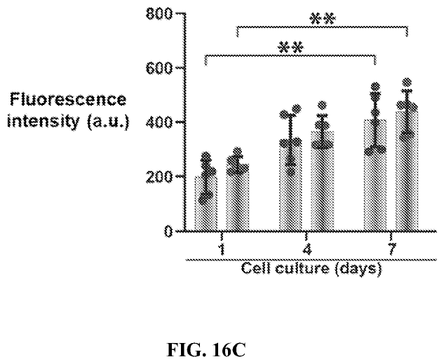

FIGS. 16A-C show biocompatibility assessment of GHS made from thermoresistant microgels. FIG. 16A shows that NIH-3T3 murine fibroblast cells were mixed with thermoresponsive or thermoresistant microgels, then photocrosslinked to form GelMA GHS or TS GelMA GHS, respectively. Fluorescent microscopy using a combination of green (representing live cells) and red (representing dead cells) dyes was performed over a period of 7 days, demonstrating cell proliferation and viability.

FIG. 16B shows the results if cell viability percentage indicated a consistently high viability rate (˜100%) for all GHS samples.

### Conclusions

Limited vascularization is a major bottleneck in reconstructive surgery and regenerative engineering. A hybrid technology utilizing a novel microsurgical approach and microporous GHS has been proposed. GHS was engineered using three different sizes of microgel building blocks to precisely tailor scaffold microarchitecture, which was used to pattern microvascular networks in vitro and in rat hindlimbs. MP was used as a facile microsurgical approach to rapidly vascularize adjacent GHS within 7 days. Accelerated and guided vascularization correlated to early macrophage and endothelial cell accumulation within MP scaffolds, showing that GHS provide a suitable base for neovascularization. Importantly, the optimum scaffold microarchitecture was identified in GHS-M, which had interconnected cell-scale pore characteristics that significantly promoted vascular network formation in terms of mean vascular density, average vessel diameter, and total tube length. In addition, the microvasculature was patterned at varying intercapillary distances using GHS with varying microgel building block sizes, and no systemic cytotoxicity was identified. It is anticipated that hybrid MP-GHS technology provides unique opportunities for physiologic neovascularization and sets up a novel translational platform for reconstructive surgery and regenerative engineering.

### Experimental Section

### Materials

Gelatin Type A from porcine skin (≈300 g Bloom), methacrylic anhydride (contains 2,000 ppm Topanol A as inhibitor, 94% purity), Dulbecco's phosphate-buffered saline (DPBS), lithium phenyl-2,4,6-trimethylbenzoylphosphinate (LAP), trichloro (1H,1H,2H,2H-perfluorooctyl) silane (F-silane), fluorescein isothiocyanate-dextran (FITC-Dextran) with an average molecular weight of 2 MDa, Hematoxylin and Eosin (H&E) staining kit, Xylenes (mixed isomers, histological grade), D-(+)-Glucose (1 M, sterile-filtered), glutaraldehyde solution (grade II, 25 vol. % in H₂O), and 1,1-dioctadecyl-3,3,3,3-tetramethylindocarbocyanine perchlorate (carbocyanine) were purchased from Sigma, MA, USA. Glass slides vacuum filtration (pore size=0.20 μm) systems, and formalin solution (10 wt. % neutral buffered) were purchased from VWR, PA, USA, and silicon wafers (4-inch) were procured from University Wafers, MA, USA. Negative photoresists were all from the KMPR 1000 series, Kayaku Advanced Materials, MA, USA. The polydimethylsiloxane (PDMS) SYLGARD® 184 kit was purchased from Dow Corning, MI, USA. Ultra-pure Milli-Q water with an electrical resistivity ≈18 MΩ at 25° C. was provided by a purification system, manufactured by the Millipore Corporation, MA, USA. Dialysis membranes with 12-14 kDa molecular weight cutoff were purchased from Spectrum Laboratories, NJ, USA. Novec 7500 Engineered Fluid (Novec oil) was provided by 3M, MN, USA. Pico-Surf (5 vol. % in Novec) was purchased from Cambridge, UK, and 1H, 1H,2H,2H-perfluoro-1-octanol (PFO) was supplied by Alfa Aeser, MA, USA. Mesenchymal stem cell growth medium 2 and endothelial cell growth medium MV were purchased from PromoCell, Germany. Hanks balanced salt solution (HBSS) was obtained from Genesse Scientific, CA. CellTracker green 5-chloromethylfluorescein diacetate (CMFDA), 100 μm and 40 μm strainers, collagenase type I, red blood cell (RBC) lysis buffer, and endothelial growth factor (EGF) module containing mucin like hormone receptor 1 (EMR1, rabbit) F4/80 polyclonal antibody were procured from ThermoFisher, MA, USA. Ethanol (absolute, anhydrous, 200 proof) was purchased from Greenfield Global, CT, USA, and isoflurane was provided by Piramel, PA, USA. Mouse platelet-endothelial cell adhesion molecule (PECAM-1/CD31) antibody. A betadine-antiseptic povidone-iodine solution was obtained from Purdue Products, CT, USA. Alexa Fluor 493 and 647 and Fluoroshield were purchased from enQuire BioReagent, CO, USA. Ficoll-Paque was acquired from GE HealthCare, PA, USA.

### Microfluidic Device Fabrication

Fabrication of high-throughput step emulsification microfluidic devices was conducted via soft lithography according to the literature. Briefly, two- or three-layered master molds were fabricated on silicon wafers using the KMPR 1000 series as the negative photoresists. The first layer was fabricated with KMPR 1005, KMPR 1025, or KMPR 1035 for small, medium, or large droplet fabrication, respectively. The spin coating condition for each mold was adopted from the manufacturer's specification sheet. The first layer height was around 8, 27, or 60 μm for small, medium, or large droplet fabrication, respectively. The second layer was deposited using the KMPR 1035 to become 2-3 times larger than the anticipated droplet size, providing enough space for forming and moving droplets. The microfluidic devices were molded using the PDMS kit. The base and crosslinker of PDMS were mixed at a 10 to 1 mass ratio, poured onto the masters, and vacuum degassed to eliminate air bubbles, followed by curing at 80° C. for 2 h. The PDMS devices were then bonded onto pre-cleaned glass slides via air plasma treatment at 400 mTorr for 45 s, followed by F-silane (2 vol. % in Novec oil) treatment to render the devices fluorophobic. Treated devices were rinsed twice with Novec oil and maintained at 80° C. for 30 min to evaporate the remaining oil.

### GelMA Polymer Synthesis

Briefly, 20 g of gelatin Type A was added to 200 mL of DPBS at 50° C. while stirring at 200 rpm. Once gelatin was fully dissolved, the reaction was initiated by adding 16 mL of methacrylic anhydride to the solution at 50° C. The solution was protected from light by wrapping the reaction container with aluminum foil. After 2 h, the reaction was stopped by adding 400 mL of DPBS. The diluted solution was then dialyzed against Milli-Q water at 40° C. for 10 days using the dialysis membranes (molecular weight cutoff=12-14 kDa). Finally, the purified solution was sterile filtered using the vacuum filtration system and maintained at −80° C. for 24 h. The frozen solution was lyophilized to yield solid GelMA. The degree of substitution was 71±3% (n=3).

### Microgel Fabrication

A GelMA polymer solution (10 wt. % in DPBS) was converted to small (diameter ≈29±3 μm), medium (≈81±4 μm), or large (≈173±11 μm) droplets via injecting the aqueous phase in a continuous oil phase using the step-emulsification microfluidic devices comprising a step size of 8, 27, or 60 μm, respectively. To prepare the aqueous phase, the photoinitiator solution containing 0.1 wt. % of LAP in DPBS was prepared, followed by dissolving GelMA at 50° C. to obtain a 10 wt. % aqueous GelMA solution. The oil phase was Novec oil, supplemented with varying concentrations of the Pico-Surf surfactant. The concentration of surfactant in the oil was 2 vol. % for the fabrication of small and medium droplets, and 0.5 vol. % for the large droplets. To avoid blockage of microfluidic channels, the droplet fabrication setup was maintained at ≈40° C. using a space heater and/or a hair dryer. The fabricated droplets were shielded from light and maintained at 4° C. overnight to form physically crosslinked GelMA microgels.

### GHS Fabrication

The oil and surfactant were removed from the physically crosslinked GelMA microgels via adding a solution of PFO (20 vol. % in Novec oil) at a 1:1 volume ratio. The suspension was then vortexed and centrifuged at 300×g for 15 s, followed by discarding the excess oil and surfactant and adding the photoinitiator solution (0.1 wt. % in DPBS) at a 1:1 volume ratio. This mixture was again vortexed, centrifuged at 300×g for 15 s, and separated from any remaining oil and surfactant. Microgels were then packed at a higher centrifugal force (3000×g), and the excess photoinitiator solution was discarded. The packed microgel suspension was transferred to an acrylic mold mounted on a glass slide using a positive displacement pipette (Microman E M100E or Microman E M1000E, Gilson, OH, USA). Packed small, medium, or large microgels were then photochemically crosslinked via light exposure (wavelength=395-405 nm, intensity=15 mW cm⁻², exposure time=60 s) to form GHS-S, GHS-M, or GHS-L, respectively.

### Bulk Hydrogel Scaffold Fabrication

Bulk GelMA hydrogel scaffolds were fabricated using a two-step crosslinking approach, similar to the GHS fabrication method to resemble the same local physicochemical properties. In brief, lyophilized GelMA was dissolved at 50° C. in the photoinitiator solution (0.1 wt. % of LAP in DPBS), yielding a clear solution with a final concentration of 10 wt. % GelMA. The GelMA solution was then pipetted out using a pre-warmed (37° C.) pipette tip and poured into laser-cut acrylic molds. The molds were maintained in a custom-built humidity chamber, protected from light, and placed at 4° C. overnight for physical crosslinking. Molded scaffolds were exposed to light (wavelength=395-405 nm, intensity=15 mW cm⁻², exposure time=60 s) to form bulk GelMA hydrogel scaffolds.

### GHS Pore Characterization

GHS were fabricated in disk molds (diameter=8 mm and height=1 mm), followed by adding ≈20 μL of the FITC-dextran solution (concentration=15 μM in DPBS) on top of them. The scaffolds were then imaged using a fluorescence microscope (Leica DMi8 THUNDERED microscope, Germany) to generate 3D Z-stacked images with a total depth of 160, 110, or 60 μm for GHS-L, GHS-M, or GHS-S, respectively. The microscope built-in software (LAS X 5.0.3 Life Science Microscope Software Platform) was used to generate 3D images from the Z-stacked images and determine the void fraction based on the ratio of space occupied by the fluorescent dye to the total volume of imaged section. A custom-developed MATLAB code was used to measure the pore characteristics by detecting the void spaces and approximating the diameter of circles that would occupy the same area.

### Compression Analysis of Scaffolds

GelMA GHS comprising varying sizes of microgel building blocks and bulk hydrogel scaffolds (as a control group, containing 10 wt. % of GelMA polymer) were fabricated in disk molds (diameter=8 mm and height=1 mm). Compression tests were performed using an Instron mechanical tester (Instron 5542, MA, USA) at a compression rate of 1 mm min-1. To determine the compressive modulus, a linear regression was performed in the elastic region of compressive strain-stress curve, typically at strain ≈0.05-0.15 mm mm⁻¹. The slope of the regression line was reported as the compressive modulus.

### Rheological Assessments of Scaffolds

To evaluate the viscoelastic properties of GHS and bulk hydrogels, disk scaffolds (diameter=8 mm and height=1 mm) were fabricated and assessed via oscillatory rheological tests. An AR-G2 rheometer (TA instrument, DE, USA) was equipped with an 8 mm diameter sandblasted top plate and a 25 mm bottom plate to sandwich the samples at 25° C. Amplitude sweep tests were performed at strain ≈10⁻² to 10²% and a fixed frequency (1 rad s⁻¹) to determine the linear viscoelastic region (LVR) for each scaffold. Frequency sweep tests were performed at 10⁻¹ to 10² rad s⁻¹ and a constant strain (0.1%).

### Isolation of Human Adipose-Derived Stromal Vascular Fraction (SVF) Cells

Human SVF cells were obtained from discarded adipose tissue, obtained from consented patients undergoing elective lipectomy at The Pennsylvania State University (Hershey, PA) under Institutional Review Board (IRB) approval protocol (#00004972). The lipectomy tissue was cleaned using HBSS to remove blood, followed by mechanical mincing after which tissue was also enzymatically digested using 1% collagenase at 37° C. on a shaker for 2 h. Collagenase-digested tissue was centrifuged for 10 min at 300×g and the pellet was collected. The pellet was then suspended in the RBC lysis buffer to remove RBC. The pellet was further centrifuged and filtered through 100 μm and 40 μm strainers. After re-suspension in HBSS, the pellet was layered on a Ficoll-Paque gradient and centrifuged for 20 min at 300×g. The SVF cells, the white layer (middle) band, were identified and collected.

### In Vitro Human SVF Cell Culture

Human SVF cells were cultured in the complete mesenchymal stem cell growth media at 37° C. with 5 vol. % carbon dioxide (CO₂) gas. Media were changed every other day. The cells were cultured and maintained to reach 80% confluency and were fluorescently labeled using the CellTracker CMFDA dye according to the manufacturer protocol. Then, the cells (passage four) and microgels were mixed using the positive displacement pipette to yield 4×10³ cells per μL of microgel suspension. To fabricate cell-laden bulk hydrogel scaffolds, the cells were suspended in a pre-gel solution, and the resulting suspension was used to fabricate the scaffold. The cell-laden GHS and bulk hydrogel controls were fabricated and cultured in the endothelial cell growth medium MV for two days. After 2 days, cells were imaged using a fluorescence microscope (Leica DMi8 THUNDERED microscope, Germany) with 10× magnification (region of interest=1330×1330 μm²), and analyzed using Ibidi Tube Formation AI Analysis (Meta Vi Labs, TX, USA) and Fiji ImageJ software (1.53t, NIH, MD, USA) for number of branches and roundness/projected area, respectively.

### Precision MP in a Rat Hindlimb Model

Animal surgery was performed at Penn State Hershey Medical Center in concordance with an Institute Animal Care and Use Committee (IACUC) approved protocol (#47941). Sprague-Dawley (SD) rats around the age of 12 weeks were used (Charles River, MA, USA). Rats were anesthetized with isoflurane, and the surgical site was shaved and prepped with betadine solution. Incisions were made on the inner aspect of each hindlimb, exposing the femoral artery and vein and allowing for circumferential vessel dissection over a length of 15 mm. A 60 μm needle was used to create 15 MP at 1 mm intervals into the femoral artery and vein. The contralateral hindlimb was surgically manipulated in an identical manner but without MP. Tested conditions were MP (n=12 hindlimbs) and no MP (control; n=12 hindlimbs). Three rats were used for each GHS-S, GHS-M, GHS-L, or bulk (control) hydrogel group. The GHS-S, GHS-M, GHS-L, and bulk (control) hydrogel scaffolds were prepared in a custom-made acrylic mold of 15 mm (length)×10 mm (width)×3 mm (thickness). The scaffolds were directly placed on the exposed femoral artery and vein. Buried absorbable sutures were used for skin closure. Rats were placed in individual cages and housed in a standard day/night light cycle environment with ad libitum food and water. Carprofen was used as a single one-time subcutaneous injection for pain control. Animals were euthanized on Day 7 for further analysis.

### Immunofluorescence Staining

Immunofluorescence staining was conducted to determine cellular infiltration in the scaffolds (n=3 scaffolds per condition). Anti-CD31 and anti-F4/80 antibodies were used to detect endothelial cells and macrophages, respectively. To prepare the tissue slides for immunofluorescence, they were deparaffinized and rehydrated by immersion in xylene and ethanol, each two times for 10 min. Next, slides were immersed in ethanol 95 vol. % for 5 min, followed by immersion in 70 and 50 vol. % alcohol for 5 min. They were rinsed with deionized water and prepared for antibody staining, as previously described. Secondary antibodies labeled with AlexaFluor 493 or 647 were used. Samples were mounted and DAPI counterstained with Fluroshield. Images were captured using the EVOS FL Auto Imaging System (ThermoFisher Scientific, MA, USA). Ten images at 20× magnification (region of interest=582×436 μm²) were used from each group for the AI analysis. AI analysis has been shown to provide accurate quantification of vascular features. Ibidi Tube Formation AI Analysis (Meta Vi Labs, TX, USA) was used for CD31 quantification and vessel analysis. Labeled images were used to train the AI to detect large blood vessels. These multiple training sessions were completed with Ibidi prior to final analysis to ensure accurate vessel density measurements. The total tube length, average vessel diameter, number of branch points, and mean vascular density of the CD31 positively labeled cells were calculated (see FIG. 11). The intercapillary distance and immune cell staining area were counted using the Fiji ImageJ software (1.53t, NIH, MD, USA). A total of 24 animals, with six hindlimbs per group, were used for detailed analysis at Day 7.

### Demonstration of Perfusability

At Day 7, in vivo perfusion assessment (n=3 scaffolds per condition or GHS-M versus Bulk) was conducted using a previously described fluorescence vessel painting technique. Under general anesthesia, an aortotomy was made just proximal to the iliac bifurcation. An olive-tipped catheter (Medtronic DLP™ 1.8″ internal mammary artery Cannula, 1 mm tip, Dublin, Ireland) was then inserted and secured in place. The lower extremity vasculature was perfused with 50-100 mL of 37° C. DPBS. The inferior vena cava was transected to allow outflow. After DPBS flushing, the lower extremity vasculature was fixed by injecting 40 mL of 2.5 vol. % glutaraldehyde solution. Adequate fixation was confirmed by the observation of nail bed pallor and stiffening of the rodent tail. Immediately after, each limb was perfused with carbocyanine. This lipophilic carbocyanine tracer had been previously dissolved in ethanol (6 mg mL-1) and stored at 4° C. as a 6.42 mM stock solution. Just prior to infusion, the stock solution was diluted 50 times in PBS, containing glucose (200 mM) to a final dye concentration of 0.128 mM. Once again, adequate distal infusion of the vasculature was inferred by a pink color change in the hind nail beds and pink fluid extravasation from the transected inferior vena cava. Scaffolds were then explanted en bloc with the underlying femoral vessels. Special care was taken to remove any adherent muscle fibers. The explants were placed between two glass slides and fixed in 10 wt. % formalin for 48 h. Specimens were rinsed briefly in distilled water before being permanently mounted with a DAPI containing mounting medium.

### Histological Evaluation

Day 7 tissue samples from the liver, spleen, and kidney (n=3 per condition) were taken from both control and MP GHS-M. Thin sections of the tissues were cut from formalin fixed paraffin embedded tissue blocks. After deparaffinization and rehydration, the sample slides underwent H&E staining. Blinded and random images of the control and MP sections were taken using the EVOS FL Auto Imaging System (Thermo Fisher Scientific, Waltham, MA). Samples were assessed for normalcy via comparison with the native rat liver, spleen, and kidney characteristics.

### Statistical Analyses

To ensure the reliability and validity of the data, a large sample size, multiple measurement points, strict inclusion and exclusion criteria, and appropriate controls and blinding was used. One-way or two-way analysis of variance (ANOVA) was performed on the study groups, followed by Tukey's post-hoc multiple comparison test, using GraphPad Prism (9.5.0, San Diego, CA). To determine the level of significancy, Student's t-test to analyze the number of branches in the in vitro test between GHS-S and GHS-M was used. All the data were acquired with at least three iterations (n≥3). The levels of significance were stated with ns: non-significant, p>0.05, *p<0.05, **p<0.01, ***p<0.001, ****p<0.0001.

### REFERENCES

Each of the following references is incorporated herein by reference in its entirety.
1. M. J. Bosse, E. J. Mackenzie, J. F. Kellam, A. R. Burgess, L. X. Webb, M. F. Swiontkowski, R. W. Sanders, A. L. Jones, M. P. McAndrew, B. M. Patterson, M. L. McCarthy, T. G. Travison, R. C. Castillo, An Analysis of Outcomes of Reconstruction or Amputation after Leg-Threatening Injuries. N. Engl. J. Med. 347, 1924-1931 (2002).
2. C. Karimkhani, R. P. Dellavalle, Luc, E. Coffeng, C. Flohr, R. J. Hay, S. M. Langan, E. O. Nsoesie, A. J. Ferrari, Holly, E. Erskine, J. I. Silverberg, T. Vos, M. Naghavi, Global skin disease morbidity and mortality an update from the global burden of disease study 2013. JAMA Dermatology 153, 406-412 (2017).
3. D. L. Horn, J. Shen, E. Roberts, T. N. Wang, K. S. Li, G. E. O'Keefe, J. Cuschieri, E. M. Bulger, B. R. H. Robinson, Predictors of mortality, limb loss, and discharge disposition at admission among patients with necrotizing skin and soft tissue infections. J. Trauma Acute Care Surg. 89, 186-191 (2020).
4. R. W. Murphree, Impairments in Skin Integrity Nurs. Clin. North Am. 52, 405-417 (2017).
5. C. Holmes, J. Wrobel, M. P. Mac Eachern, B. R. Boles, Collagen-based wound dressings for the treatment of diabetes-related foot ulcers: a systematic review. Diabetes, Metab. Syndr. Obes. Targets Ther. 6, 17 (2013).
6. F. Pallaske, A. Pallaske, K. Herklotz, J. Boese-Landgraf, The significance of collagen dressings in wound management: a review. J. Wound Care 27, 692-702 (2018).
7. D. A. Banyard, J. M. Bourgeois, A. D. Widgerow, G. R. D. Evans, Regenerative Biomaterials: A Review. Plast. Reconstr. Surg. 135, 1740-1748 (2015).
8. J. J. Kim, G. R. D. Evans, Applications of Biomaterials in Plastic Surgery. Clin. Plast. Surg. 39, 359-376 (2012).
9. H. Mehdizadeh, S. Sumo, E. S. Bayrak, E. M. Brey, A. Cinar, Three-dimensional modeling of angiogenesis in porous biomaterial scaffolds. Biomaterials 34, 2875-2887 (2013).
10. E. Dantzer, F. M. Braye, Reconstructive surgery using an artificial dermis (Integra): results with 39 grafts. Br. J. Plast. Surg. 54, 659-664 (2001).
11. Y. F. Diehm, S. Fischer, E. Gazyakan, G. Hundeshagen, D. Kotsougiani-Fischer, F. Falkner, U. Kneser, C. Hirche, Negative pressure wound therapy as an accelerator and stabilizer for incorporation of artificial dermal skin substitutes—A retrospective, non-blinded, and non-randomized comparative study. J. Plast. Reconstr. Aesthetic Surg. 74, 357-363 (2021).
12. P. C. Hancock, S. V. Koduru, M. Sun, D. J. Ravnic, Induction of scaffold angiogenesis by recipient vasculature precision micropuncture. Microvasc. Res. 134, 104121 (2021).
13. J. Rouwkema, N. C. Rivron, C. A. van Blitterswijk, Vascularization in tissue engineering. Trends Biotechnol. 26, 434-441 (2008).
14. L. J. Chen, H. Kaji, Modeling angiogenesis with micro- and nanotechnology. Lab Chip 17, 4186-4219 (2017).
15. A. Sheikhi, J. de Rutte, R. Haghniaz, O. Akouissi, A. Sohrabi, D. Di Carlo, A. Khademhosseini, Microfluidic-enabled bottom-up hydrogels from annealable naturally-derived protein microbeads. Biomaterials 192, 560-568 (2019).
16. Z. Ataie, S. Kheirabadi, J. W. Zhang, A. Kedzierski, C. Petrosky, R. Jiang, C. Vollberg, A. Sheikhi, Nanoengineered Granular Hydrogel Bioinks with Preserved Interconnected Microporosity for Extrusion Bioprinting. Small 18, 2202390 (2022).
17. A. C. Daly, L. Riley, T. Segura, J. A. Burdick, Hydrogel microparticles for biomedical applications. Nat. Rev. Mater. 5, 20-43 (2020).
18. L. Riley, L. Schirmer, T. Segura, Granular hydrogels: emergent properties of jammed hydrogel microparticles and their applications in tissue repair and regeneration. Curr. Opin. Biotechnol. 60, 1-8 (2019).
19. Y. Kang, J. Chang, Channels in a porous scaffold: a new player for vascularization. Regen. Med. 13, 705-715 (2018).
20. P. Xia, Y. Luo, Vascularization in tissue engineering: The architecture cues of pores in scaffolds. J. Biomed. Mater. Res. Part B Appl. Biomater. 110, 1206-1214 (2022).
21. Y.-C. Chiu, M.-H. Cheng, H. Engel, S.-W. Kao, J. C. Larson, S. Gupta, E. M. Brey, The role of pore size on vascularization and tissue remodeling in PEG hydrogels. Biomaterials 32, 6045-6051 (2011).
22. T. H. Qazi, L. Tytgat, P. Dubruel, G. N. Duda, S. Van Vlierberghe, S. Geissler, Extrusion Printed Scaffolds with Varying Pore Size As Modulators of MSC Angiogenic Paracrine Effects. ACS Biomater. Sci. Eng. 5, 5348-5358 (2019).
23. L. Zieber, S. Or, E. Ruvinov, S. Cohen, Microfabrication of channel arrays promotes vessel-like network formation in cardiac cell construct and vascularization in vivo. Biofabrication 6, 024102 (2014).
24. F. Bai, Z. Wang, J. Lu, J. Liu, G. Chen, R. Lv, J. Wang, K. Lin, J. Zhang, X. Huang, The Correlation Between the Internal Structure and Vascularization of Controllable Porous Bioceramic Materials In Vivo: A Quantitative Study. Tissue Eng. Part A 16, 3791-3803 (2010).
25. T. Yu, Q. Liu, T. Jiang, X. Wang, Y. Yang, Y. Kang, Channeled β-TCP Scaffolds Promoted Vascularization and Bone Augmentation in Mandible of Beagle Dogs. Adv. Funct. Mater. 26, 6719-6727 (2016).
26. D. B. Kolesky, K. A. Homan, M. A. Skylar-Scott, J. A. Lewis, Three-dimensional bioprinting of thick vascularized tissues. Proc. Natl. Acad. Sci. 113, 3179-3184 (2016).
27. L. Shao, Q. Gao, C. Xie, J. Fu, M. Xiang, Y. He, Directly coaxial 3D bioprinting of large-scale vascularized tissue constructs. Biofabrication 12, 035014 (2020).
28. S. I. Somo, B. Akar, E. S. Bayrak, J. C. Larson, A. A. Appel, H. Mehdizadeh, A. Cinar, E. M. Brey, Pore Interconnectivity Influences Growth Factor-Mediated Vascularization in Sphere-Templated Hydrogels. Tissue Eng. Part C Methods 21, 773-785 (2015).
29. M. A. Traore, S. C. George, Tissue Engineering the Vascular Tree. Tissue Eng.—Part B Rev. 23, 505-514 (2017).
30. V. Mastrullo, W. Cathery, E. Velliou, P. Madeddu, P. Campagnolo, Angiogenesis in Tissue Engineering: As Nature Intended? Front. Bioeng. Biotechnol. 8, 1-13 (2020).
31. A. Sheikhi, J. de Rutte, R. Haghniaz, O. Akouissi, A. Sohrabi, D. Di Carlo, A. Khademhosseini, Modular microporous hydrogels formed from microgel beads with orthogonal thermo-chemical responsivity: Microfluidic fabrication and characterization. MethodsX 6, 1747-1752 (2019).
32. N. Zoratto, D. Di Lisa, J. Rutte, M. N. Sakib, A. R. Alves e Silva, A. Tamayol, D. Di Carlo, A. Khademhosseini, A. Sheikhi, In situ forming microporous gelatin methacryloyl hydrogel scaffolds from thermostable microgels for tissue engineering. Bioeng. Transl. Med. 5 (2020).
33. C.-C. Hua, X.-M. Liu, L.-R. Liang, L.-F. Wang, J.-C. Zhong, Targeting the microRNA-34a as a Novel Therapeutic Strategy for Cardiovascular Diseases. Front. Cardiovasc. Med. 8, 1-11 (2022).
34. J. M. de Rutte, J. Koh, D. Di Carlo, Scalable High-Throughput Production of Modular Microgels for In Situ Assembly of Microporous Tissue Scaffolds. Adv. Funct. Mater. 29, 1-10 (2019).
35. K. Yue, G. Trujillo-de Santiago, M. M. Alvarez, A. Tamayol, N. Annabi, A. Khademhosseini, Synthesis, properties, and biomedical applications of gelatin methacryloyl (GelMA) hydrogels. Biomaterials 73, 254-271 (2015).
36. Y. Wang, R. K. Kankala, C. Ou, A. Chen, Z. Yang, Advances in hydrogel-based vascularized tissues for tissue repair and drug screening. Bioact. Mater. 9, 198-220 (2022).
37. R. W. Barrs, J. Jia, S. E. Silver, M. Yost, Y. Mei, Biomaterials for Bioprinting Microvasculature. Chem. Rev. 120, 10887-10949 (2020).
38. K. Yue, X. Li, K. Schrobback, A. Sheikhi, N. Annabi, J. Leijten, W. Zhang, Y. S. Zhang, D. W. Hutmacher, T. J. Klein, A. Khademhosseini, Structural analysis of photocrosslinkable methacryloyl-modified protein derivatives. Biomaterials 139, 163-171 (2017).
39. Y. Zhang, P. Kumar, S. Lv, D. Xiong, H. Zhao, Z. Cai, X. Zhao, Recent advances in 3D bioprinting of vascularized tissues. Mater. Des. 199, 109398 (2021).
40. H. Bae, A. F. Ahari, H. Shin, J. W. Nichol, C. B. Hutson, M. Masaeli, S. H. Kim, H. Aubin, S. Yamanlar, A. Khademhosseini, Cell-laden microengineered pullulan methacrylate hydrogels promote cell proliferation and 3D cluster formation. Soft Matter 7, 1903-1911 (2011).
41. H. Aubin, J. W. Nichol, C. B. Hutson, H. Bae, A. L. Sieminski, D. M. Cropek, P. Akhyari, A. Khademhosseini, Directed 3D cell alignment and elongation in microengineered hydrogels. Biomaterials 31, 6941-6951 (2010).
42. J. Ramón-Azcón, S. Ahadian, R. Obregón, G. Camci-Unal, S. Ostrovidov, V. Hosseini, H. Kaji, K. Ino, H. Shiku, A. Khademhosseini, T. Matsue, Gelatin methacrylate as a promising hydrogel for 3D microscale organization and proliferation of dielectrophoretically patterned cells. Lab Chip 12, 2959 (2012).
43. S. Lee, J. De Rutte, R. Dimatteo, D. Koo, D. Di Carlo, Scalable Fabrication and Use of 3D Structured Microparticles Spatially Functionalized with Biomolecules. ACS Nano 16, 38-49 (2022).
44. S. Han, H. M. Sun, K. C. Hwang, S. W. Kim, Adipose-derived stromal vascular fraction cells: Update on clinical utility and efficacy. Crit. Rev. Eukaryot. Gene Expr. 25, 145-152 (2015).
45. Sigma-Aldrich, FITC-labelled polysaccharides (available at https://www.sigmaaldrich.com/technical-documents/articles/chemistry/fluorescently-labeled-dextrane.html).
46. C. F. Guimarães, L. Gasperini, A. P. Marques, R. L. Reis, The stiffness of living tissues and its implications for tissue engineering. Nat. Rev. Mater. 5, 351-370 (2020).
47. S. Budday, T. C. Ovaert, G. A. Holzapfel, P. Steinmann, E. Kuhl, Fifty Shades of Brain: A Review on the Mechanical Testing and Modeling of Brain Tissue. Arch. Comput. Methods Eng. 27, 1187-1230 (2020).
48. A. Forghani, S. V. Koduru, C. Chen, A. N. Leberfinger, D. J. Ravnic, D. J. Hayes, Differentiation of Adipose Tissue-Derived CD34+/CD31− Cells into Endothelial Cells In Vitro. Regen. Eng. Transl. Med. 6, 101-110 (2020).
49. J. G. Maijub, N. L. Boyd, J. R. Dale, J. B. Hoying, M. E. Morris, S. K. Williams, Concentration-Dependent Vascularization of Adipose Stromal Vascular Fraction Cells. Cell Transplant. 24, 2029-2039 (2015).
50. KMPR® 1000|Kayaku Advanced Materials, Inc. (available at https://kayakuam.com/products/kmpr-1000/).
51. Z. Ataie, A. Jaberi, S. Kheirabadi, A. Risbud, A. Sheikhi, Gelatin Methacryloyl Granular Hydrogel Scaffolds: High-throughput Microgel Fabrication, Lyophilization, Chemical Assembly, and 3D Bioprinting. J. Vis. Exp. 190, e64829 (2022).
52. S. V. Koduru, A. N. Leberfinger, I. T. Ozbolat, D. J. Ravnic, Navigating the Genomic Landscape of Human Adipose Stem Cell-Derived β-Cells. Stem Cells Dev. 30, 1153-1170 (2021).
53. P. A. Zuk, M. Zhu, H. Mizuno, J. Huang, J. W. Futrell, A. J. Katz, P. Benhaim, H. P. Lorenz, M. H. Hedrick, Multilineage cells from human adipose tissue: Implications for cell-based therapies. Tissue Eng. 7, 211-228 (2001).
54. P. A. Zuk, M. Zhu, P. Ashjian, D. A. De Ugarte, J. I. Huang, H. Mizuno, Z. C. Alfonso, J. K. Fraser, P. Benhaim, M. H. Hedrick, M. Raff, Ed. Human adipose tissue is a source of multipotent stem cells. Mol. Biol. Cell 13, 4279-4295 (2002).
55. T. T. Hormel, T. S. Hwang, S. T. Bailey, D. J. Wilson, D. Huang, Y. Jia, Artificial intelligence in OCT angiography. Prog. Retin. Eye Res. 85, 100965 (2021).
56. Fiji Downloads (available at https://imagej.net/software/fiji/downloads).
57. D. J. Ravnic, X. Jiang, T. Wolloscheck, J. P. Pratt, H. Huss, S. J. Mentzer, M. A. Konerding, Vessel painting of the microcirculation using fluorescent lipophilic tracers. Microvasc. Res. 70, 90-96 (2005).

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the apparatus and methods of using and making the same disclosed herein have been discussed and illustrated, it is to be distinctly understood that the invention is not limited thereto but may be otherwise variously embodied and practiced within the scope of the following claims.

1. A method for regenerating tissue and/or inducing vascularization, the method comprising:
optionally perforating a wall of a target vessel; and
implanting a granular scaffold on a target tissue and/or the target vessel.

2. The method of claim 1, further comprising:
prior to implanting the granular scaffold on the target tissue and/or the target vessel,
converting polymers to form microparticles via physical and/or chemical crosslinking; and
assembling the microparticles to form the granular scaffold via physical and/or chemical bond formation.

3. The method of claim 2, wherein a shape of the microparticles is selected from the group consisting of spheres, cubes, cuboids, cones, cylinders, pyramids, and prisms.

4. The method of claim 2, wherein the microparticles are selected from the group consisting of hydrogels, plastics, elastomers, thermosets, and other polymers.

5. The method of claim 2, further comprising:
injecting the microparticles at the target tissue and/or the target vessel prior to assembling the microparticles.

6. The method of claim 2, wherein the polymers are selected from the group consisting of proteins, peptides, carbohydrates, lipids, and mixtures thereof.

7. The method of claim 6, wherein the polymer is gelatin methacryloyl.

8. The method of claim 1, wherein the granular scaffold has a mean pore diameter in the order of a cell size of the target tissue and/or the target vessel.

9. The method of claim 1, wherein the granular scaffold has a median pore diameter of 0.1-1000 μm, and wherein the granular scaffold has a mean void fraction between 20-25%.

10. (canceled)

11. The method of claim 2, wherein the microparticles adhere to 1-10,000 cells.

12. The method of claim 1, wherein the granular scaffold has a tissue adhesion strength of 1-500 kPa.

13. The method of claim 1, wherein the granular scaffold is configured to have pro-angiogenic cell signaling properties.

14. The method of claim 1, wherein the method is configured to facilitate vascularization of the granular scaffold within 1-90 days of implanting the granular scaffold on the target tissue and/or the target vessel.

15. The method of claim 1, wherein the method is configured to facilitate vascularization of the granular scaffold within 7-28 days of implanting the granular scaffold on the target tissue and/or the target vessel.

16. The method of claim 1, wherein the step of perforating the wall of the target vessel comprises perforating the wall with a needle with a diameter of 1-2000 μm.

17. The method of claim 1, wherein the granular scaffold is porous with or without the use of microparticles.

18. The method of claim 1, wherein the granular scaffold is prefabricated or in situ fabricated on the target tissue and/or the target vessel.

19. The method of claim 1, wherein the granular scaffold is additively manufactured via 3D printing.

20. The method of claim 1, wherein the granular scaffold is used for the regeneration, repair, reconstruction, and/or closure of soft and/or hard tissues with or without the step of perforating the wall of the target vessel.

21. A method for inducing and/or patterning hierarchical vascularization, the method comprising:
implanting a granular scaffold on a target tissue and/or a target vessel; and
perforating a wall of the target vessel.
